SEQUENCE LISTING (142 sequences)

SEQ ID NO: 1 - protein, 115 aa, synthetic construct
QVQLVQSGAEVKKPGASVKVSCKASGFNIKDYYIHWVRQAPGQGLEWIGWIDPENGDTEYAPKFQGRATMTADTSISTAYMELSRLRSDDTAVYYCNAGGRFAYWGQGTLVTVSS

SEQ ID NO: 2 - protein, 113 aa, synthetic construct
DIQMTQSPSSLSASVGDRVTITCKSSQSLLNSGNQKNYLAWYQQKPGKAPKLLIYGASTRESGVPSRFSGSGSGTDFTLTISSLQPEDFATYYCQNDHSYPYTFGGGTKLEIK

SEQ ID NO: 3 - protein, 115 aa, synthetic construct
QVQLVQSGAEVKKPGASVKVSCKASGFNIKDYYIHWVRQATGQGLEWMGWIDPENGDPEYAQKFQGRVTMTRDTSINTAYMELSSLRSEDTAVYYCNAGGRFAYWGQGTLVTVSS

SEQ ID NO: 4 - protein, 113 aa, synthetic construct
DIQMTQSPSSLSASVGDRVTITCRASQSLLNSGNQKNYLAWYQQKPGKVPKLLIYGASTRESGVPSRFSYSGSGTDFTLTISSLQPEDVWTYYCQNDHSYPYTFGQGTKLEIK

SEQ ID NO: 5 - protein, 115 aa, synthetic construct; identical to SEQ ID NO: 3

SEQ ID NO: 6 - protein, 113 aa, synthetic construct
DIVMTQSPDSLAVSLGERATINCKSSQSLLNSGNQKNYLAWYQQKPGQPWKLLIYGASTRESGVPDRFSGSGSGTDFTLTISSLQAEDVYVYYCQNDHSYPYTFFQGTKVEIK

SEQ ID NO: 7 - protein, 115 aa, synthetic construct; identical to SEQ ID NO: 1

SEQ ID NO: 8 - protein, 113 aa, synthetic construct
DIQMTQSPSTLSASVGDRVTITCKSSQSLLNSGNQKNYLAWYQQKPGKAPKLLIYGASTRESGVPSRFSGSGSGTEFTLTISSLQPDDFATYYCQNDHSYPYTFGGGTKVEIK

SEQ ID NO: 9 - protein, 16 aa, unidentified
SSGGGGSGGGGSGGGG

SEQ ID NO: 10 - protein, 115 aa, synthetic construct
EVQLQQAGAELVRSGASVKLSCTASGFNIKDYYIHWVKQRPEQGLEWIGWIDPENGDTEYAPKFQGKATMTADTSSNTAYLQLSSLTSEDTAVYYCNAGGRFAYWGQGTLVTVSA

SEQ ID NO: 11 - protein, 113 aa, synthetic construct
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPYTFGGGTKLEIK

SEQ ID NO: 12 - protein, 115 aa, synthetic construct
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 13 - protein, 95 aa, synthetic construct
QPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 14 - protein, 115 aa, synthetic construct; identical to SEQ ID NO: 12

SEQ ID NO: 15 - protein, 95 aa, synthetic construct; identical to SEQ ID NO: 13

SEQ ID NO: 16-21 - skipped (6 entries)

SEQ ID NO: 22 - protein, 16 aa, synthetic construct
GGGGSGGGGSGGGGSS

SEQ ID NO: 23-30 - skipped (8 entries)

SEQ ID NO: 31 - protein, 8 aa, synthetic construct
GFNIKDYY

SEQ ID NO: 32 - protein, 8 aa, synthetic construct
IDPENGDT

SEQ ID NO: 33 - protein, 8 aa, synthetic construct
NAGGRFAY

SEQ ID NO: 34 - protein, 12 aa, synthetic construct
QSLLNSGNQKNY

SEQ ID NO: 35 - skipped

SEQ ID NO: 36 - protein, 9 aa, synthetic construct
QNDHSYPYT

SEQ ID NO: 37-40 - skipped (4 entries)

SEQ ID NO: 41 - protein, 178 aa, synthetic construct
QVQLVQSGAEVKKPGASVKVSCKASIHWVRQAPGQGLEWIGEYAPKFQGRATMTADTSISTAYMELSRLRSDDTAVYYCWGQGTLVTVSSDIQMTQSPSSLSASVGDRVTITCKSSLAWYQQKPGKAPKLLITRESGVPSRFSGSGSGTDFTLTISSLQPEDFATYYCFGGGTKLEIK

SEQ ID NO: 42 - protein, 180 aa, synthetic construct
QVQLVQSGAEVKKPGASVKVSCKASIHWVRQATGQGLEWMGWEYAQKFQGRVTMTRDTSINTAYMELSSLRSEDTAVYYCWGQGTLVTVSSDIQMTQSPSSLSASVGDRVTITCRASLAWYQQKPGKVPKLLIYTRESGVPSRFSYSGSGTDFTLTISSLQPEDVWTYYCFGQGTKLEIK

SEQ ID NO: 43 - protein, 178 aa, synthetic construct
QVQLVQSGAEVKKPGASVKVSCKASIHWVRQATGQGLEWMGWEYAQKFQGRVTMTRDTSINTAYMELSSLRSEDTAVYYCWGQGTLVTVSSDIVMTQSPDSLAVSLGERATINCKSLAWYQQKPGQPWKLLIYTRESGVPDRFSGSGSGTDFTLTISSLQAEDVYVYYFFQGTKVEIK

SEQ ID NO: 44 - protein, 179 aa, synthetic construct
QVQLVQSGAEVKKPGASVKVSCKASIHWVRQAPGQGLEWIGWYAPKFQGRATMTADTSISTAYMELSRLRSDDTAVYYCWGQGTLVTVSSDIQMTQSPSTLSASVGDRVTITCKSSLAWYQQKPGKAPKLLIYTRESGVPSRFSGSGSGTEFTLTISSLQPDDFATYYCFGGGTKVEIK

SEQ ID NO: 45-49 - skipped (5 entries)

SEQ ID NO: 50 - protein, 15 aa, Homo sapiens
CETPRIRGTGTRELE

SEQ ID NO: 51 - protein, 25 aa, synthetic construct [sequence omitted]

SEQ ID NO: 52 - protein, 16 aa, synthetic construct
IHWVRQAPGQGLEWIG

SEQ ID NO: 53 - protein, 38 aa, synthetic construct
EYAPKFQGRATMTADTSISTAYMELSRLRSDDTAVYYC

SEQ ID NO: 54 - protein, 11 aa, synthetic construct
WGQGTLVTVSS

SEQ ID NO: 55 - protein, 26 aa, synthetic construct
DIQMTQSPSSLSASVGDRVTITCKSS

SEQ ID NO: 56 - protein, 16 aa, synthetic construct
LAWYQQKPGKAPKLLI

SEQ ID NO: 57 - protein, 36 aa, synthetic construct
TRESGVPSRFSGSGSGTDFTLTISSLQPEDFATYYC

SEQ ID NO: 58 - protein, 10 aa, synthetic construct
FGGGTKLEIK

SEQ ID NO: 59 - protein, 25 aa, synthetic construct [sequence omitted]

SEQ ID NO: 60 - protein, 17 aa, synthetic construct
IHWVRQATGQGLEWMGW

SEQ ID NO: 61 - protein, 38 aa, synthetic construct
EYAQKFQGRVTMTRDTSINTAYMELSSLRSEDTAVYYC

SEQ ID NO: 62 - protein, 11 aa, synthetic construct
WGQGTLVTVSS

SEQ ID NO: 63 - protein, 26 aa, synthetic construct [sequence omitted]

SEQ ID NO: 64 - protein, 17 aa, synthetic construct
LAWYQQKPGKVPKLLIY

SEQ ID NO: 65 - protein, 36 aa, synthetic construct
TRESGVPSRFSYSGSGTDFTLTISSLQPEDVWTYYC

SEQ ID NO: 66 - protein, 10 aa, synthetic construct
FGQGTKLEIK

SEQ ID NO: 67 - protein, 25 aa, synthetic construct [sequence omitted]

SEQ ID NO: 68 - protein, 17 aa, synthetic construct
IHWVRQATGQGLEWMGW

SEQ ID NO: 69 - protein, 38 aa, synthetic construct; identical to SEQ ID NO: 61

SEQ ID NO: 70 - protein, 11 aa, synthetic construct
WGQGTLVTVSS

SEQ ID NO: 71 - protein, 25 aa, synthetic construct
DIVMTQSPDSLAVSLGERATINCKS

SEQ ID NO: 72 - protein, 17 aa, synthetic construct
LAWYQQKPGQPWKLLIY

SEQ ID NO: 73 - protein, 35 aa, synthetic construct
TRESGVPDRFSGSGSGTDFTLTISSLQAEDVYVYY

SEQ ID NO: 74 - protein, 10 aa, synthetic construct
FFQGTKVEIK

SEQ ID NO: 75 - protein, 25 aa, synthetic construct [sequence omitted]

SEQ ID NO: 76 - protein, 17 aa, synthetic construct
IHWVRQAPGQGLEWIGW

SEQ ID NO: 77 - protein, 37 aa, synthetic construct
YAPKFQGRATMTADTSISTAYMELSRLRSDDTAVYYC

SEQ ID NO: 78 - protein, 11 aa, synthetic construct
WGQGTLVTVSS

SEQ ID NO: 79 - protein, 26 aa, synthetic construct
DIQMTQSPSTLSASVGDRVTITCKSS

SEQ ID NO: 80 - protein, 17 aa, synthetic construct
LAWYQQKPGKAPKLLIY

SEQ ID NO: 81 - protein, 36 aa, synthetic construct
TRESGVPSRFSGSGSGTEFTLTISSLQPDDFATYYC

SEQ ID NO: 82 - protein, 10 aa, synthetic construct
FGGGTKVEIK

SEQ ID NO: 83-89 - skipped (7 entries)

SEQ ID NO: 90 - protein, 263 aa, synthetic construct
MAQVQLVQSGAEVKKPGASVKVSCKASGFNIKDYYIHWVRQAPGQGLEWIGWIDPENGDTEYAPKFQGRATMTADTSISTAYMELSRLRSDDTAVYYCNAGGRFAYWGQGTLVTVSSGGGGSGGGGSGGGGSDIQMTQSPSSLSASVGDRVTITCKSSQSLLNSGNQKNYLAWYQQKPGKAPKLLIYGASTRESGVPSRFSGSGSGTDFTLTISSLQPEDFATYYCQNDHSYPYTFGGGTKLEIKENLYFQGAAALEHHHHHH

SEQ ID NO: 91 - protein, 263 aa, synthetic construct; identical to SEQ ID NO: 90

SEQ ID NO: 92 - protein, 263 aa, synthetic construct; identical to SEQ ID NO: 90

SEQ ID NO: 93 - protein, 263 aa, synthetic construct
MAQVQLVQSGAEVKKPGASVKVSCKASGFNIKDYYIHWVRQAPGQGLEWIGWIDPENGDTEYAPKFQGRATMTADTSISTAYMELSRLRSDDTAVYYCNAGGRFAYWGQGTLVTVSSGGGGSGGGGSGGGGSDIQMTQSPSTLSASVGDRVTITCKSSQSLLNSGNQKNYLAWYQQKPGKAPKLLIYGASTRESGVPSRFSGSGSGTEFTLTISSLQPDDFATYYCQNDHSYPYTFGGGTKVEIKENLYFQGAAALEHHHHHH

SEQ ID NO: 94 - protein, 263 aa, synthetic construct; identical to SEQ ID NO: 90

SEQ ID NO: 95 - skipped

SEQ ID NO: 96 - protein, 210 aa, synthetic construct
MAQVQLVQSGAEVKKPGASVKVSCKASIHWVRQAPGQGLEWIGWEYAPKFQGRATMTADTSISTAYMELSRLRSDDTAVYYCWGQGTLVTVSSWGQGTLVTVSSLINKERDIQMTQSPSSLSASVGDRVTITCKSSLAWYQQKPGKAPKLLIYTRESGVPSRFSGSGSGTDFTLTISSLQPEDFATYYFGGGTKLEIKENLYFQGAAALE

SEQ ID NO: 97 - protein, 202 aa, synthetic construct
MAQVQLVQSGAEVKKPGASVKVSCCDRHCIHWVRQAPGQGLEWIGWYAPKFQGRATMTADTSISTAYMELSRLRSDDTAVYYCNAGGRFAYWGQGTLVTVSSDIQMTQSPSSLSASVGDRVTITCKSSLAWYQQKPGKAPKLLIYTRESGVPSRFSGSGSGTDFTLTISSLQPEDFATYYFGGGTKLEIKENLYFQGAAALE

SEQ ID NO: 98-99 - skipped (2 entries)

SEQ ID NO: 100 - protein, 26 aa, synthetic construct
DIQMTQSPSSLSASVGDRVTITCKSS

SEQ ID NO: 101 - protein, 17 aa, synthetic construct
LAWYQQKPGKAPKLLIY

SEQ ID NO: 102 - protein, 36 aa, synthetic construct; identical to SEQ ID NO: 57

SEQ ID NO: 103 - protein, 22 aa, synthetic construct
FGGGTKLEIKENLYFQGAAALE

SEQ ID NO: 104 - protein, 27 aa, synthetic construct
MAQVQLVQSGAEVKKPGASVKVSCKAS

SEQ ID NO: 105 - protein, 17 aa, synthetic construct
IHWVRQAPGQGLEWIGW

SEQ ID NO: 106 - protein, 38 aa, synthetic construct; identical to SEQ ID NO: 53

SEQ ID NO: 107 - protein, 11 aa, synthetic construct
WGQGTLVTVSS

SEQ ID NO: 108 - protein, 26 aa, synthetic construct
DIQMTQSPSTLSASVGDRVTITCKSS

SEQ ID NO: 109 - protein, 17 aa, synthetic construct
LAWYQQKPGKAPKLLIY

SEQ ID NO: 110 - protein, 35 aa, synthetic construct
RESGVPSRFSGSGSGTEFTLTISSLQPDDFATYYC

SEQ ID NO: 111 - protein, 22 aa, synthetic construct
FGGGTKVEIKENLYFQGAAALE

SEQ ID NO: 112-119 - skipped (8 entries)

SEQ ID NO: 120 - protein, 115 aa, synthetic construct
MAQVQLVQSGAEVKKPGASVKVSCKASGFNIKDYYIHWVRQAPGQGLEWIGWIDPENGDTEYAPKFQGRATMTADTSISTAYMELSRLRSDDTAVYYCNAGGRFAYWGQGTLVTV

SEQ ID NO: 121 - protein, 125 aa, synthetic construct
DIQMTQSPSSLSASVGDRVTITCKSSQSLLNSGNQKNYLAWYQQKPGKAPKLLIYGASTRESGVPSRFSGSGSGTDFTLTISSLQPEDFATYYCQNDHSYPYTFGGGTKLEIKENLYFQGAAALE

SEQ ID NO: 122 - protein, 125 aa, synthetic construct
DIQMTQSPSTLSASVGDRVTITCKSSQSLLNSGNQKNYLAWYQQKPGKAPKLLIYGASTRESGVPSRFSGSGSGTEFTLTISSLQPDDFATYYCQNDHSYPYTFGGGTKVEIKENLYFQGAAALE

SEQ ID NO: 123 - protein, 27 aa, synthetic construct
MAQVQLVQSGAEVKKPGASVKVSCKAS

SEQ ID NO: 124 - protein, 17 aa, synthetic construct
IHWVRQAPGQGLEWIGW

SEQ ID NO: 125 - protein, 38 aa, synthetic construct; identical to SEQ ID NO: 53

SEQ ID NO: 126 - protein, 9 aa, synthetic construct
WGQGTLVTV

SEQ ID NO: 127 - protein, 26 aa, synthetic construct
DIQMTQSPSSLSASVGDRVTITCKSS

SEQ ID NO: 128 - protein, 17 aa, synthetic construct
LAWYQQKPGKAPKLLIY

SEQ ID NO: 129 - protein, 36 aa, synthetic construct; identical to SEQ ID NO: 57

SEQ ID NO: 130 - protein, 22 aa, synthetic construct
FGGGTKLEIKENLYFQGAAALE

SEQ ID NO: 131 - protein, 26 aa, synthetic construct
DIQMTQSPSTLSASVGDRVTITCKSS

SEQ ID NO: 132 - protein, 17 aa, synthetic construct
LAWYQQKPGKAPKLLIY

SEQ ID NO: 133 - protein, 36 aa, synthetic construct; identical to SEQ ID NO: 81

SEQ ID NO: 134 - protein, 22 aa, synthetic construct
FGGGTKVEIKENLYFQGAAALE

SEQ ID NO: 135 - protein, 245 aa, synthetic construct
MAQVQLVQSGAEVKKPGASVKVSCKASGFNIKDYYIHWVRQAPGQGLEWIGWIDPENGDTEYAPKFQGRATMTADTSISTAYMELSRLRSDDTAVYYCNAGGRFAYWGQGTLVTVSSGGGGSGGGGSGGGGSDIQMTQSPSSLSASVGDRVTITCKSSQSLLNSGNQKNYLAWYQQKPGKAPKLLIYGASTRESGVPSRFSGSGSGTDFTLTISSLQPEDFATYYCQNDHSYPYTFGGGTKLEIK

SEQ ID NO: 136 - protein, 245 aa, synthetic construct; identical to SEQ ID NO: 135

SEQ ID NO: 137 - protein, 245 aa, synthetic construct; identical to SEQ ID NO: 135

SEQ ID NO: 138 - protein, 245 aa, synthetic construct
MAQVQLVQSGAEVKKPGASVKVSCKASGFNIKDYYIHWVRQAPGQGLEWIGWIDPENGDTEYAPKFQGRATMTADTSISTAYMELSRLRSDDTAVYYCNAGGRFAYWGQGTLVTVSSGGGGSGGGGSGGGGSDIQMTQSPSTLSASVGDRVTITCKSSQSLLNSGNQKNYLAWYQQKPGKAPKLLIYGASTRESGVPSRFSGSGSGTEFTLTISSLQPDDFATYYCQNDHSYPYTFGGGTKVEIK

SEQ ID NO: 139 - protein, 245 aa, synthetic construct; identical to SEQ ID NO: 135

SEQ ID NO: 140 - protein, 115 aa, synthetic construct; identical to SEQ ID NO: 120

SEQ ID NO: 141 - protein, 125 aa, synthetic construct; identical to SEQ ID NO: 121

SEQ ID NO: 142 - protein, 125 aa, synthetic construct; identical to SEQ ID NO: 122

(19) United States
(12) Patent Application Publication
(10) Pub. No.: US 2025/0313643 A1
(43) Pub. Date: Oct. 9, 2025

(54) HUMANIZED NON-OPIOID COMPOSITION AND THERAPIES FOR PAIN MANAGEMENT

(71) Applicants: UNM Rainforest Innovations, Albuquerque, NM (US); Mayo Foundation for Medical Education and Research, Rochester, MN (US); United States Department of Veterans Affairs, Washington, DC (US)

(72) Inventors: Karin Westlund High, Albuquerque, NM (US); Adinarayana Kunamneni, Jacksonville, FL (US); Sascha R.A. ALLES, Albuquerque, NM (US)

(21) Appl. No.: 18/872,217

(22) PCT Filed: Jun. 6, 2023
(86) PCT No.: PCT/US2023/068000

§ 371 Date: Dec. 5, 2024

Related U.S. Application Data
(60) Provisional application No. 63/349,772, filed on Jun. 7, 2022.

(51) Int. Cl. C07K 16/28; A61K 9/00; A61K 39/00; A61P 25/02; A61P 25/22; A61P 25/24; C12N 15/86

(52) U.S. Cl. CPC C07K 16/2869; A61K 9/0019; A61P 25/02; A61P 25/22; A61P 25/24; C12N 15/86; A61K 2039/505; A61K 2039/54; C07K 2317/24; C07K 2317/55; C07K 2317/565; C07K 2317/567; C07K 2317/622

(57) ABSTRACT

Antibodies or portions thereof specific for human CCKBR, a composition comprising the antibody or portion thereof, an isolated cell expressing the antibody or portion thereof, and a method to inhibit or treat pain by using the antibody or portion thereof, are provided.

### CROSS-REFERENCE TO RELATED APPLICATIONS

This application claims the benefit of the filing date of U.S. application 63/349,772, filed on Jun. 7, 2022, the disclosure of which is incorporated by reference herein.

### STATEMENT OF GOVERNMENT RIGHTS

The invention was made with government support under grants DE028096 and HEAL UG3 NS123958 awarded by the National Institutes of Health (NIH). The Government has certain rights in the invention.

### INCORPORATION BY REFERENCE OF SEQUENCE LISTING

A Sequence Listing is provided herewith as an xml file, “2340673.xml” created on Jun. 6, 2023, and having a size of 102,504 bytes. The content of the xml file is incorporated by reference herein in its entirety.

### BACKGROUND

Traumatic blunt force injuries can directly injure and sensitize the trigeminal nerve innervating the head, dura, and tooth sockets, or other nerves such as the sciatic nerve in the leg. A serious consequence of nerve injury pain or “neuropathic pain” injuries is that some can transition from acute to chronic pain. The persisting nerve injury can generate additional mechanisms centrally in the nervous system, creating nerve overactivation and molecular alterations along the brain's pain circuitry, referred to as “central sensitization”. Chronic pain is comorbid in 70% of patients with Traumatic Brain Injury (TBI) in part due to direct peripheral nerve damage. Likewise, 22% of non-battle blunt force trauma nerve injuries sustained to head, face, and neck are most often due to motor vehicle accidents.

### SUMMARY

The disclosure provides a humanized non-opioid antibody, e.g., a small antibody, therapy for, for example, chronic pain, e.g., induced by inflammatory and/or nerve injury. A panel of small murine single-chain variable fragment (scFv) antibodies recognizing a peptide of CCKBR (CCKB receptor is a cholecystokinin B receptor) were generated with cell-free ribosome display technology. The scFv antibodies feature binding activity similar to monoclonal antibodies but with stronger affinity and increased tissue penetrability due to their smaller size.

Based on the success of the murine parent scFv scFv77-2, a panel of humanized scFvs that bind with specificity to an extracellular peptide of mouse CCK-BR (also referred to as cholecystokinin 2 receptor, CCKBR, CCK2R, or gastrin receptor), CETPRIRGTGTRELE (SEQ ID NO:50), was generated using transient production in Rosetta Gami/CHO cells and protein purification. The extracellular fragment of human CCK-BR (CETPRIRGTGTRELE; SEQ ID NO: 50), corresponds to amino acid residues 39-53 of mouse

Gastrin/cholecystokinin type B receptor. The human CCKBR peptide has 13/15 amino acid residues identical to mouse. Three distinct variable heavy and three variable light chains were selected, and can be combined to make a total of nine distinct heavy and light chain combinations. Using the T20 values of these humanized scFv77-2 variants, very low immunogenicity is expected in patients. Affinity measurement by ELISA indicates binding affinity in the low nanomolar range, in comparison to the murine parental. The HC2-LC3 had more than 25-fold improvement in affinity compared with parental mscFv77-2. In vivo validation found reversal of pain related behaviors within one to two weeks after a single dose (e.g., 4 mg/kg, intraperitoneal, subcutaneous, or intranasal). This constitutes a method whereby reversal of pain can be accomplished by providing treatment with a humanized scFv, e.g., to reverse the effect of nerve injury activation of CCKBR that promotes a cascade of events culminating in chronic pain. In addition, the humanized CCKBR scFv prevents the development of anxiety-and depression-like behaviors and stress typical in week 6-8 in the untreated mice with persisting pain-like behaviors in chronic pain models. Thus, the scFvs are useful to inhibit or treat chronic pain, e.g., neuropathic pain, such as that experienced in trigeminal neuralgia, sciatica, back pain, diabetes, PTSD, and multiple sclerosis. That is, the scFvs, which have binding activity like monoclonal antibodies, a stronger binding affinity, and increased tissue penetrability. For example, they are brain/nervous tissue penetrant due to their smaller size, and so a single dose may permanently alleviate chronic pain as shown in 3 nerve injury models, or prevent anxiety-and depression-like behaviors and stress. In one embodiment, the scFv is intranasally administered. In one embodiment, the scFv is subcutaneously administered. In one embodiment, 1 mg/kg to 10 mg/kg, e.g., 3 mg/kg to 5 mg/kg such as 4 mg/kg. These doses are for mice but typically a dose/kg is equivalent in many species of the scFv is administered subcutaneously or intranasally in mice.

In one embodiment, the scFVs, which bind to, e.g., inhibit or block, CCKBR, can relieve pain-or anxiety-related behavior, and/or return neuronal firing to baseline while reducing inflammatory mediators, e.g., in chronic pain mouse models, and so can be employed to inhibit or treat neuropathic pain, nerve injury, e.g., of the trigeminal nerve, hypersensitivity, allodynia, and prevent anxiety, stress or depression in a mammal. Repeated treatment may be even more effective.

In one embodiment, a composition comprising an anti-human CCKBR antibody, or an antigen binding fragment thereof, or a polypeptide, that prevents or inhibits human CCKBR activity is provided, where the antibody, the antigen binding fragment thereof, or the polypeptide has a variable immunoglobulin (Ig) region comprising at least one of GFNIKDYY (SEQ ID NO:31), IDPENGDT (SEQ ID NO:32), NAGGRFAY (SEQ ID NO:33), QSLLNSGNQKNY (SEQ ID NO: 34), GAS or QNDHSYPYT (SEQ ID NO:36), or any combination thereof. In one embodiment, antibody is a scFv. In one embodiment, antibody is a single domain antibody, e.g., a nanobody, such as one having only the variable region of a heavy chain of an antibody including one that is humanized. A humanized antibody or fragment thereof may be formed of human variable region sequences excluding one of more of the CDRs (DRs), where one or more of the CDRs are a consensus sequence or from a non-human mammal.

In one embodiment, an isolated cell comprising an expression cassette comprising a heterologous promoter operably linked to nucleic acid sequences encoding an anti-human CCKBR antibody, or an antigen binding fragment thereof, or a polypeptide, that prevents or inhibits human CCKBR activity is provided, where the antibody, the antigen binding fragment thereof, or the polypeptide has an amino acid sequence comprising at least one of GFNIKDYY (SEQ ID NO:31), IDPENGDT (SEQ ID NO:32), NAGGRFAY (SEQ ID NO: 33), QSLLNSGNQKNY (SEQ ID NO:34), GAS or QNDHSYPYT (SEQ ID NO: 36), or any combination thereof. In one embodiment, the cell is a mammalian cell, e.g., a primate cell such as a human cell. In one embodiment, the cell is a plant cell. In one embodiment, the cell is an insect cell.

In one embodiment, an isolated nucleic acid comprising a promoter operably linked to a nucleotide sequence which encodes at least the variable region of a heavy or light Ig chain that binds human CCKBR, is provided wherein the chain comprises at least one of GFNIKDYY (SEQ ID NO:31), IDPENGDT (SEQ ID NO:32), NAGGRFAY (SEQ ID NO:33), QSLLNSGNQKNY (SEQ ID NO:34), GAS or QNDHSYPYT (SEQ ID NO:36), or any combination thereof. In one embodiment, a scFv is administered.

Further provided is a method to inhibit or treat stress, depression, or anxiety in a mammal, comprising: administering to a mammal a composition comprising an effective amount of a nucleotide sequence which encodes at least the variable region of a heavy or light chain that binds human CCKBR, wherein the chain comprises at least one of GFNIKDYY (SEQ ID NO:31), IDPENGDT (SEQ ID NO:32), NAGGRFAY (SEQ ID NO:33), QSLLNSGNQKNY (SEQ ID NO: 34), GAS or QNDHSYPYT (SEQ ID NO:36), or any combination thereof. In one embodiment, the mammal is a human. In one embodiment, the composition is systemically administered. In one embodiment, the composition is injected. In one embodiment, the nucleotide sequence is in a viral vector. In one embodiment, the composition is locally administered. In one embodiment, the composition is intranasally administered. The cognitive disruption associated with stress was also alleviated by the treatment of a stress disorder.

Also provided is a method to prevent, inhibit or treat pain in a mammal, comprising: administering to a mammal a composition comprising an effective amount of a nucleotide sequence which encodes at least the variable region of a heavy or light chain that binds human CCKBR, wherein the chain comprises at least one of GFNIKDYY (SEQ ID NO:31), IDPENGDT (SEQ ID NO:32), NAGGRFAY (SEQ ID NO:33), QSLLNSGNQKNY (SEQ ID NO:34), GAS or QNDHSYPYT (SEQ ID NO:36), or any combination thereof. In one embodiment, the mammal has acute pain. In one embodiment, the mammal has chronic pain. In one embodiment, the mammal has neuropathic pain. In one embodiment, the mammal was exposed to blunt force trauma. In one embodiment, the mammal has traumatic brain injury. In one embodiment, the mammal is a human. In one embodiment, the composition is systemically administered. In one embodiment, the composition is injected. In one embodiment, the nucleotide sequence is in a viral vector. In one embodiment, the composition is locally administered. In one embodiment, the composition is administered intranasally.

Thus, the compositions disclosed herein may be useful to prevent, inhibit or treat chronic pain, acute pain, nerve injury pain, back pain, muscle pain, neuropathic pain, trigeminal neuralgia, multiple sclerosis, PTSD, or diabetes pain, while preventing associated anxiety, stress, depression, and cognitive disruption. In one embodiment, the composition is orally administered. In one embodiment, the composition is a tablet. In one embodiment, the composition is a capsule. In one embodiment, the composition is injected. In one embodiment, the composition is intranasally administered. In one embodiment, the composition is subcutaneously administered. In one embodiment, the composition comprises a buffer.

### BRIEF DESCRIPTION OF FIGURES

FIG. 1A. Schematic of generation of antibodies and initial binding studies. The alignment shows that there is approximately 88% homology between mouse and human CCK-B receptors.

FIG. 1B. Binding affinity of four variants and parental antibody.

FIG. 1C is a schematic of monoclonal antibody and scFv. Humanization by CDR-grafting includes transferring parental (commonly rodent) complementarity determining regions (CDR) into human framework (FR) regions. Parental antibody specificity and affinity are conserved thanks to the preservation of residues implicated in antigen binding. A modernized version of the classical CDR-grafting technology was employed.

FIG. 2. Humanized CCKBR scFv Variants Relieve Mechanical Hypersensitivity/Mechanical Threshold Testing with von Frey Fibers on the face. Threshold increase indicates alleviation of hypersensitivity. 4 of the 5 variants tested had alleviated orofacial pain related behaviors in the FRICT-ION trigeminal chronic neuropathic pain mouse model. n-6. Binding affinity is indicated (Kd).

FIG. 3. Humanized CCKBR scFv Variants Relieve Cold Hypersensitivity. All 4 variants tested alleviated orofacial cold pain related behaviors Cold Probe testing on the face (−10° C.). FRICT-ION trigeminal chronic neuropathic pain mouse model. Increase in time before withdrawal indicates alleviation of hypersensitivity

FIG. 4A. Four humanized CCKBR scFv Variants Reverse Anxiety Measure. FRICT-ION trigeminal chronic neuropathic pain mouse model. Less time is spent in normal exploratory behavior. Vehicle treated mice with unrelieved orofacial pain prefer to stay in the dark. Sucrose spritzed on the tail of mice results in grooming in normal mice and mice with FRICT-ION treated with 3 of 4 CCK-BR scFvs (FIG. 4B).

FIG. 5. Dose-response efficacy testing with selected humanized CCKBR HC2-LC3 scFv. FRICT-ION trigeminal chronic neuropathic pain mouse model, n=4. Von Frey; mechanical threshold increase; cold probe temperature relief (minus 10° C.). Two routes of administration were employed, subcutaneous and intranasal.

FIG. 6. Comparator drug treatment with CCKBR Inhibitor, LY225910. Efficacy in FRICT-ION model (Treated vs Naïve Controls). Mice were given 10 mg/kg LY225910 daily (subcutaneous injection). On Day 0, mice were tested hourly to see the hour of highest efficacy. On days 1-8, mice were tested prior to dose, then two hours after dose to confirm efficacy and see if baseline changed with paradigm. Effects of LY225910 versus treatment with HC2-LC3 CCKBR scFv. Results match treatment with HC2-LC3 (n=4). Mechanical sensitivity was evaluated using the von Frey filament test at D1-8 after treatment in BALBc mice with FRICT-ION. Increased threshold indicates less pain. n=3 mice per group ***p<0.001 vs FRICT-ION+vehicle.

FIG. 7. Effect of CCKBR hscFv HC2-LC3 on CCKBR Immunostaining in Mouse DRG. Naïve mice have low levels of CCKBR protein immunostaining. FRICT-ION significantly increases the CCKBR immunostaining (10 weeks post). Treatment of FRICT-ION mice with a single dose of CCKBR hscFv HC2-LC3 in week 3 restores the baseline content of CCKBR immunostaining (week 10 post).

FIG. 8. Spared nerve injury (SNI) chronic neuropathic pain model. Humanized scFv HC2-LC3 effectively relieves hindpaw mechanical and heat hypersensitivity.

FIG. 9. Back pain model (urokinase lumbar ligament injection model). Humanized scFv HC2-LC3 effectively reduces hindpaw mechanical and heat hypersensitivity in males and females.

FIG. 10. hscFv HC2-LC3 protected mice from predator stress. Chronic predator urine stress test daily 10 min exposure for 3 weeks, 1.5 weeks after hscFv HC2-LC3 dosing. Light/Dark Anxiety Test (n=8).

FIG. 11A. Novel Object recognition test demonstrates the HC2-LC3 restores the cognitive disruption that is found in untreated mice with chronic pain. FIG. 11B. Mechanical threshold testing with von Frey fibers in the FRICTION trigeminal chronic neuropathic pain mouse model. Pilot multidosing study done with murine CCK-BR scFv 77-2. indicates better efficacy with multidosing.

FIG. 12. Rheobase with human donor cells in the presence or absence of humanized scFv. Rheobase is the minimum amount of current needed to elicit firing. Effect of HC2-LC3 hCCKBR-scFv (LEAD) on human DRG neuron excitability. A significant increase in rheobase of hDRG neurons was observed following 10 μg/ml, 1 hr treatment with hCCKBR scFv variant HC2-LC3 (LEAD). Cells from 3 donors (one 22 yo female, one 51 yo female and one 23 yo male) combined. Control n=50, HC2-LC3 scFv n=41. *p<0.05, **p<0.01 Mann-Whitney test.

FIG. 13. Humanized CCKBR scFv variants reduce firing frequency of mouse DRG neurons. Firing frequency vs. current injection (above rheobase) following treatment for 1 hr with 10 μg/ml of scFv (n=14 untreated, n=14 HC2-LC1, n=16. HC1-LC2 and n=10 HC2-LC3) compared to control. One-way ANOVA with Tukey's multiple comparisons test, Control vs. HC2-LC1, p<0.05*, Control vs. HC1-LC2, p<0.0001, Control vs. HC2-LC3, p<0.0001.

FIG. 14. Heat maps showing upregulated (red) and downregulated (blue) differentially expressed genes (5-fold and less) in trigeminal ganglia of vehicle treated FRICT-ION mice and mice treated with the humanized CCKBR scFvHC2-LC3. Trigeminal nerve-injured mice with the FRICT-ION were treated 3 weeks after induction of injury with humanized CCKBR scFv. Trigeminal ganglia were collected 7 weeks after hscFv treatment. The heat map indicates an overall decrease (blue) in gene expression (fold >5) in the trigeminal ganglia (TG) of mice with FRICTION treated with humanized CCKBR scFv compared to untreated FRICT-ION mice with chronically injured trigeminal nerves (week 10; 7 weeks after hscFv treatment). The TG of untreated mice had one microRNA increased (m467f, 2.97-fold). The m467f are reported to be involved in the cellular response to tumor necrosis factor. The mice treated with humanized CCKBR scFv had decreased expression of four microRNAs (m142hg, −2.88; m320, −3.70; m466i, −3.07; m6386, −3.69). The microRNA 142hg and 320 have been associated with gene silencing in genomic databases. The m142 has been associated with inflammation in the literature. Data of others has suggested that m320 is involved in neuronal regeneration. The m466i has been associated with cellular response to amino acid stimulus.

### DETAILED DESCRIPTION

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double-and single-stranded molecules. Unless otherwise specified or required, any embodiment of the disclosure described herein that is a polynucleotide encompasses both the double-stranded form and each of two complementary single-stranded forms known or predicted to make up the double-stranded form.

“Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double-and single-stranded DNA, and double-and single-stranded RNA. The terms include, as equivalents, analogs of either RNA or DNA made from nucleotide analogs and modified polynucleotides such as, though not limited to, methylated and/or capped polynucleotides.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this disclosure are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present disclosure generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevents transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence.

This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present disclosure are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. For example, the mouse and human CCKBR amino acid sequence generating the mouse scFv is 88% homologous with the human amino acid sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

The term “antibody,” as used herein, may refer to a full-length immunoglobulin molecule or an immunologically-active fragment of an immunoglobulin molecule such as the Fab or F(ab′)2 fragment generated by, for example, cleavage of the antibody with an enzyme such as pepsin or co-expression of an antibody light chain and an antibody heavy chain in, for example, a mammalian cell, or ScFv. The antibody can also be an IgG, IgD, IgA, IgE or IgM antibody. Full-length immunoglobulin “light chains” (about 25 kD or 214 amino acids) are encoded by a variable region gene at the amino-terminus (about 110 amino acids) and a kappa or lambda constant region gene at the carboxy-terminus. Full-length immunoglobulin “heavy chains” (about 50 kD or 446 amino acids), are similarly encoded by a variable region gene (about 116 amino acids) and one of the other aforementioned constant region genes, e.g., gamma (encoding about 330 amino acids). Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. In each pair of the tetramer, the light and heavy chain variable regions are together responsible for binding to an antigen, and the constant regions are responsible for the antibody effector functions. In addition to naturally occurring antibodies, immunoglobulins may exist in a variety of other forms including, for example, Fv, ScFv, Fab, and F(ab′)2, as well as bifunctional hybrid antibodies (e.g., Lanzavecchia et al. (1987)) and in single chains (e.g., Huston et al. (1988) and Bird et al. (1988), which are incorporated herein by reference). (See, generally, Hood et al., “Immunology”, Benjamin, N.Y., 2nd ed. (1984), and Hunkapiller and Hood (1986), which are incorporated herein by reference). Thus, the term “antibody” includes antigen binding antibody fragments, as are known in the art, including Fab, Fab2, single chain antibodies (scFv for example), chimeric antibodies, etc., either produced by the modification of whole antibodies or those synthesized de novo using recombinant DNA technologies.

An immunoglobulin light or heavy chain variable region consists of a “framework” region interrupted by three hypervariable regions, also called CDR's. The extent of the framework region and CDR's have been precisely defined (see, “Sequences of Proteins of Immunological Interest,” E. Kabat et al., U.S. Department of Health and Human Services, (1983); which is incorporated herein by reference). The sequences of the framework regions of different light or heavy chains are relatively conserved within a species. As used herein, a “human framework region” is a framework region that is substantially identical (about 85% or more, usually 90 to 95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. One example of a chimeric antibody is one composed of the variable or antigen-binding domain from a mouse antibody and the constant or effector domain from a human antibody, although other mammalian species may be used.

As used herein, the term “humanized” immunoglobulin refers to an immunoglobulin having a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they are generally substantially identical to human immunoglobulin constant regions, i.e., at least about 85-90%, or about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin. One says that the donor antibody has been “humanized”, by the process of “humanization”, because the resultant humanized antibody is expected to bind to the same antigen as the donor antibody that provides the CDR's.

Thus, humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody has substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will include at least a portion of an immunoglobulin constant region (Fc), typically that of a human immunoglobulin (Jones et al. (1986); Riechmann et al. (1988); and Presta (1992)).

It is understood that the humanized antibodies may have additional conservative amino acid substitutions which have substantially no effect on antigen binding or other immunoglobulin functions. By conservative substitutions are intended combinations such as gly, ala; val, ile, leu; asp, glu; asn, gln; ser, thr; lys, arg; and phe, tyr.

Humanized immunoglobulins, including humanized antibodies, have been constructed by means of genetic engineering. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321: 522 (1986); Riechmann et al., Nature, 332: 323 (1988); Verhoeyen et al., Science, 239: 1534 (1988)), by substituting rodent CDRs or CDR sequences for the corresponding sequences of a human antibody. Accordingly, such “humanized” antibodies are chimeric antibodies that have substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies.

Human antibodies can also be produced using various techniques known in the art, including phage and ribosome display libraries (Hoogenboom and Winter, J. Mol. Biol., 227: 381 (1991); Marks et al., J. Mol. Biol., 222: 581 (1991); Kunamneni et al., PlosOne. PMID: 30444865; Kunamneni et al., Am J Trop Med Hyg. PMID: 31074409). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147: 86 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779 (1992); Lonberg et al., Nature, 368: 856 (1994); Morrison, Nature, 368: 812 (1994); Fishwild et al., Nature Biotechnology, 14: 845 (1996); Neuberger, Nature Biotechnology, 14: 826 (1996); Lonberg and Huszar, Intern. Rev. Immunol., 13: 65 (1995). Most humanized immunoglobulins that have been previously described have a framework that is identical to the framework of a particular human immunoglobulin chain and three CDR's from a non-human donor immunoglobulin chain.

A framework may be one from a particular human immunoglobulin that is unusually homologous to the donor immunoglobulin to be humanized, or a consensus framework derived from many human antibodies. For example, comparison of the sequence of a mouse heavy (or light) chain variable region against human heavy (or light) variable regions in a data bank (for example, the National Biomedical Research Foundation Protein Identification Resource) shows that the extent of homology to different human regions varies greatly, typically from about 40% to about 60-70%. By choosing one of the human heavy (respectively light) chain variable regions that is most homologous to the heavy (respectively light) chain variable region of the other immunoglobulin, fewer amino acids will be changed in going from the one immunoglobulin to the humanized immunoglobulin. The precise overall shape of a humanized antibody having the humanized immunoglobulin chain may more closely resemble the shape of the donor antibody, also reducing the chance of distorting the CDR's.

Typically, one of the 3-5 most homologous heavy chain variable region sequences in a representative collection of at least about 10 to 20 distinct human heavy chains is chosen as acceptor to provide the heavy chain framework, and similarly for the light chain. One of the 1 to 3 most homologous variable regions may be used. The selected acceptor immunoglobulin chain may have at least about 65% homology in the framework region to the donor immunoglobulin.

In many cases, it may be considered desirable to use light and heavy chains from the same human antibody as acceptor sequences, to be sure the humanized light and heavy chains will make favorable contacts with each other. Regardless of how the acceptor immunoglobulin is chosen, higher affinity may be achieved by selecting a small number of amino acids in the framework of the humanized immunoglobulin chain to be the same as the amino acids at those positions in the donor rather than in the acceptor.

Humanized antibodies generally have advantages over mouse or in some cases chimeric antibodies for use in human therapy: because the effector portion is human, it may interact better with the other parts of the human immune system (e.g., destroy the target cells more efficiently by complement-dependent cytotoxicity (CDC) or antibody-dependent cellular cytotoxicity (ADCC)); the human immune system should not recognize the framework or constant region of the humanized antibody as foreign, and therefore the antibody response against such an antibody should be less than against a totally foreign mouse antibody or a partially foreign chimeric antibody.

DNA segments having immunoglobulin sequences typically further include an expression control DNA sequence operably linked to the humanized immunoglobulin coding sequences, including naturally-associated or heterologous promoter regions. Generally, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the humanized light chains, heavy chains, light/heavy chain dimers or intact antibodies, binding fragments or other immunoglobulin forms may follow (see, S. Beychok, Cells of Immunoglobulin Synthesis, Academic Press, New York, (1979), which is incorporated herein by reference).

Other “substantially homologous” modified immunoglobulins to the native sequences can be readily designed and manufactured utilizing various recombinant DNA techniques well known to those skilled in the art. For example, the framework regions can vary at the primary structure level by several amino acid substitutions, terminal and intermediate additions and deletions, and the like. Moreover, a variety of different human framework regions may be used singly or in combination as a basis for the humanized immunoglobulins of the present disclosure. In general, modifications of the genes may be readily accomplished by a variety of well-known techniques, such as site-directed mutagenesis (see, Gillman and Smith, Gene, 8: 81 (1979) and Roberts et al., Nature, 328: 731 (1987), both of which are incorporated herein by reference). Substantially homologous immunoglobulin sequences are those which exhibit at least about 85% homology, usually at least about 90%, or at least about 95% homology with a reference immunoglobulin protein.

Alternatively, polypeptide fragments comprising only a portion of the primary antibody structure may be produced, which fragments possess one or more immunoglobulin activities (e.g., antigen binding). These polypeptide fragments may be produced by proteolytic cleavage of intact antibodies by methods well known in the art, or by inserting stop codons at the desired locations in vectors known to those skilled in the art, using site-directed mutagenesis.

### Exemplary Gene Transfer Vectors

The disclosure also provides a gene transfer vector comprising a nucleic acid sequence which encodes an antibody, an antigen binding fragment thereof, or a polypeptide, directed against CCKBR. In one embodiment, the gene transfer vector is a virus. The disclosure further provides a method of using the gene transfer vector or encoded gene product against CCKBR in a mammal, which method comprises administering to the mammal the above-described gene transfer vector or the encoded gene product. Various aspects of the gene transfer vector, antibody or antigen binding fragment thereof, and methods are discussed below. Although each parameter is discussed separately, the gene transfer vector, antibody or antigen binding fragment thereof, or polypeptide, and method, may comprise combinations of the parameters set forth below. Accordingly, any combination of parameters can be used according to the gene transfer vector, antibody or antigen binding fragment thereof, the polypeptide, and the method.

A “gene transfer vector” is any molecule or composition that has the ability to carry and deliver a heterologous nucleic acid sequence into a suitable host cell where synthesis of the encoded protein takes place. Typically, a gene transfer vector is a nucleic acid molecule that has been engineered, using recombinant DNA techniques that are known in the art, to incorporate the heterologous nucleic acid sequence. Desirably, the gene transfer vector is comprised of DNA. Examples of suitable DNA-based gene transfer vectors include plasmids and viral vectors. However, gene transfer vectors that are not based on nucleic acids, such as liposomes, are also known and used in the art. The gene transfer vector can be based on a single type of nucleic acid (e.g., a plasmid) or non-nucleic acid molecule (e.g., a lipid or a polymer). The gene transfer vector can be integrated into the host cell genome, or can be present in the host cell in the form of an episome.

In one embodiment, the gene transfer vector is a viral vector. Suitable viral vectors include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Any viral vector may be employed to deliver antibody encoding sequences to cells including mammalian cells, or to mammals, include but are not limited to adeno-associated virus, adenovirus, herpesvirus, retrovirus, or lentivirus vectors. Other agents including linked agent(s) may be combined with the scFv, where the other agent(s), e.g., linked agent(s), has/have a similar or different function.

In addition to the nucleic acid sequence encoding an antibody against CCKBR, or an antigen-binding fragment thereof, the viral vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, CA. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27: 4324 (1999); Nuc. Acid. Res., 28: e99 (2000); U.S. Pat. No. 7,112,715; and Kramer & Fussenegger, Methods Mol. Biol., 308: 123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer wsequences. Enhancers can be located upstream, within, or downstream of coding sequences. In one embodiment, the nucleic acid sequence encoding an antibody against CCKBR, or an antigen-binding fragment thereof, is operably linked to a CMV enhancer/chicken beta-actin promoter (also referred to as a “CAG promoter”) (see, e.g., Niwa et al., Gene, 108: 193 (1991); Daly et al., Proc. Natl. Acad. Sci. U.S.A., 96: 2296 (1999); and Sondhi et al., Mol. Ther., 15: 481 (2007)).

Typically AAV vectors are produced using well characterized plasmids. For example, human embryonic kidney 293T cells are transfected with one of the transgene specific plasmids and another plasmid containing the adenovirus helper and AAV rep and cap genes (specific to AAVrh. 10, 8 or 9 as required). After 72 hours, the cells are harvested and the vector is released from the cells by five freeze/thaw cycles. Subsequent centrifugation and benzonase treatment removes cellular debris and unencapsidated DNA. Iodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1× phosphate buffered saline. The viral titers may be measured by TaqMan® real-time PCR and the viral purity may be assessed by SDS-PAGE.

### Exemplary CCKBR Sequences

The antibodies or fragments thereof, or polypeptides, that bind to or inhibit CCKBR, e.g., bind to the extracellular portion thereof, may bind to a polypeptide having

```
  (SEQ ID NO: 12)
mnsgvclcvl mavlaagalt qpvppadpag sglqraeeap rrqlrvsqrt dgesrahlga
llaryiqgar kapsgrmsiv knlqnldpsh risdrdymgw mdfgrrsaee yeyps;
  (SEQ ID NO: 13)
qpvppadpag sglqraeeap rrqlrvsqrt dgesrahlga llaryiqgar kapsgrmsiv
knlqnldpsh risdrdymgw mdfgrrsaee yeyps;
(SEQ ID NO: 14)
mnsgvclcvl mavlaagalt qpvppadpag sglqraeeap rrqlrvsqrt dgesrahlga
llaryiqqar kapsgrmsiv knlgnldpsh risdrdymgw mdfgrrsaee yeyps;
    (SEQ ID NO: 15)
qpvppadpag sglqraeeap rrqlrvsqrt dgesrahlga llaryiqqar kapsgrmsiv
knlqnldpsh risdrdymgw mdfgrrsaee yeyps;
```

or a polypeptide with at least 80%, 82%, 85%, 87%, 89%, 90%, 92%, 94%, 95%, 96%, 97%, 98% or 99% amino acid sequence identity thereto.
   Exemplary Humanized Anti-CCKBR scFv Sequences

In one embodiment, scFv77-2 hu HC1-LC2 may include (linker is italicized; protease site and his tag are underlined)

(SEQ ID NO: 90)
MAQVQLVQSGAEVKKPGASVKVSCKASGFNIKDYYIHWVRQAPGQGLEW
MGWIDPENGDTEYAQKFQGRVTMTADTSINTAYMELSSLRSEDTAVYYC
NAGGRFAYWGQGTLVTVSSGGGGSGGGGSGGGGSDIQMTQSPSSLSASV
GDRVTITCKSSQSLLNSGNQKNYLAWYQQKPGKAPKLLIYGASTRESGV
PSRFSGSGSGTDFTLTISSLQPEDFATYYCQNDHSYPYTFGGGTKLEIK
ENLYFQGAAALEHHHHHH*,

or a polypeptide with at least 80%, 82%, 85%, 87%, 89%, 90%, 92%, 94%, 95%, 96%, 97%, 98% or 99% amino acid sequence identity thereto.

In one embodiment, scFv77-2 hu HC2-LC1 may include

(SEQ ID NO: 91)
MAQVQLVQSGAEVKKPGASVKVSCKASGFNIKDYYIHWVRQAPGQGLEW
IGWIDPENGDTEYAPKFQGRATMTADTSISTAYMELSRLRSDDTAVYYC
NAGGRFAYWGQGTLVTVSSGGGGSGGGGSGGGGSDIVMTQSPDSLAVSL
GERATINCKSSQSLLNSGNQKNYLAWYQQKPGQPPKLLIYGASTRESGV
PDRFSGSGSGTDFTLTISSLQAEDVAVYYCQNDHSYPYTFGGGTKLEIK
ENLYFQGAAALEHHHHHH*,

or a polypeptide with at least 80%, 82%, 85%, 87%, 89%, 90%, 92%, 94%, 95%, 96%, 97%, 98% or 99% amino acid sequence identity thereto.

In one embodiment, scFv77-2 hu HC2-LC2 may include

(SEQ ID NO: 92)
MAQVQLVQSGAEVKKPGASVKVSCKASGFNIKDYYIHWVRQAPGQGLEW
IGWIDPENGDTEYAPKFQGRATMTADTSISTAYMELSRLRSDDTAVYYC
NAGGRFAYWGQGTLVTVSSGGGGSGGGGSGGGGSDIQMTQSPSSLSASV
GDRVTITCKSSQSLLNSGNQKNYLAWYQQKPGKAPKLLIYGASTRESGV
PSRFSGSGSGTDFTLTISSLQPEDFATYYCQNDHSYPYTFGGGTKLEIK
ENLYFQGAAALEHHHHHH*,

or a polypeptide with at least 80%, 82%, 85%, 87%, 89%, 90%, 92%, 94%, 95%, 96%, 97%, 98% or 99% amino acid sequence identity thereto.

In one embodiment, scFv77-2 hu HC2-LC3 may include

(SEQ ID NO: 93)
MAQVQLVQSGAEVKKPGASVKVSCKASGFNIKDYYIHWVRQAPGQGLEW
IGWIDPENGDTEYAPKFQGRATMTADTSISTAYMELSRLRSDDTAVYYC
NAGGRFAYWGQGTLVTVSSGGGGSGGGGSGGGGSDIQMTQSPSTLSASV
GDRVTITCKSSQSLLNSGNQKNYLAWYQQKPGKAPKLLIYGASTRESGV
PSRFSGSGSGTEFTLTISSLQPDDFATYYCQNDHSYPYTFGGGTKVEIK
ENLYFQGAAALEHHHHHH*,

or a polypeptide with at least 80%, 82%, 85%, 87%, 89%, 90%, 92%, 94%, 95%, 96%, 97%, 98% or 99% amino acid sequence identity thereto.

For example, scFv77-2 hu HC1-linker-LC2 may include:

### Heavy Chain

(SEQ ID NO: 1)
QVQLVQSGAEVKKPGASVKVSCKASGFNIKDYYIHWVRQAPGQGLEWMGW
IDPENGDTEYAQKFQGRVTMTADTSINTAYMELSSLRSEDTAVYYCNAGG
RFAYWGQGTLVTVSS,

or a polypeptide with at least 80%, 82%, 85%, 87%, 89%, 90%, 92%, 94%, 95%, 96%, 97%, 98% or 99% amino acid sequence identity thereto.

### Light Chain

(SEQ ID NO: 2)
DIQMTQSPSSLSASVGDRVTITCKSSQSLLNSGNQKNYLAWYQQKPGKAP
KLLIYGASTRESGVPSRFSGSGSGTDFTLTISSLQPEDFATYYCQNDHSY
PYTFGGGTKLEIK,

or a polypeptide with at least 80%, 82%, 85%, 87%, 89%, 90%, 92%, 94%, 95%, 96%, 97%, 98% or 99% amino acid sequence identity thereto.

scFv77-2 hu HC2-linker-LC1 may include:

### Heavy Chain

```
  (SEQ ID NO: 3)
QVQLVQSGAEVKKPGASVKVSCKASGFNIKDYYIHWVRQAPGQGLEWIGW
IDPENGDTEYAPKFQGRATMTADTSISTAYMELSRLRSDDTAVYYCNAGG
RFAYWGQGTLVTVSS
or
  (SEQ ID NO: 120)
MAQVQLVQSGAEVKKPGASVKVSCKASGFNIKDYYIHWVRQAPGQGLEWI
GWIDPENGDTEYAPKFQGRATMTADTSISTAYMELSRLRSDDTAVYYCNA
GGRFAYWGQGTLVTV,
```

or a polypeptide with at least 80%, 82%, 85%, 87%, 89%, 90%, 92%, 94%, 95%, 96%, 97%, 98% or 99% amino acid sequence identity thereto.

### Light Chain

```
  (SEQ ID NO: 4)
DIVMTQSPDSLAVSLGERATINCKSSQSLLNSGNQKNYLAWYQQKPGQ
PPKLLIYGASTRESGVPDRFSGSGSGTDFTLTISSLQAEDVAVYYCQND
HSYPYTFGGGTKLEIK
or
  (SEQ ID NO: 121)
DIQMTQSPSSLSASVGDRVTITCKSSQSLLNSGNQKNYLAWYQQKPGKA
PKLLIYGASTRESGVPSRFSGSGSGTDFTLTISSLQPEDFATYYCQNDH
SYPYTFGGGTKLEIKENLYFQGAAALE,
```

or a polypeptide with at least 80%, 82%, 85%, 87%, 89%, 90%, 92%, 94%, 95%, 96%, 97%, 98% or 99% amino acid sequence identity thereto. scFv77-2 hu HC2-linker-LC2 may include:

### Heavy Chain

(SEQ ID NO: 5)
QVQLVQSGAEVKKPGASVKVSCKASGFNIKDYYIHWVRQAPGQGLEWIG
WIDPENGDTEYAPKFQGRATMTADTSISTAYMELSRLRSDDTAVYYCNA
GGRFAYWGQGTLVTVSS,

85%, 87%, 89%, 90%, 92%, 94%, 95%, 96%, 97%, 98% or 99% amino acid sequence identity thereto.

### Light Chain

(SEQ ID NO: 6)
DIQMTQSPSSLSASVGDRVTITCKSSQSLLNSGNQKNYLAWYQQKPGKA
PKLLIYGASTRESGVPSRFSGSGSGTDFTLTISSLQPEDFATYYCQNDH
SYPYTFGGGTKLEIK,

or a polypeptide with at least 80%, 82%, 85%, 87%, 89%, 90%, 92%, 94%, 95%, 96%, 97%, 98% or 99% amino acid sequence identity thereto.

scFv77-2 hu HC2-linker-LC3 may include:

### Heavy Chain

(SEQ ID NO: 7)
QVQLVQSGAEVKKPGASVKVSCKASGFNIKDYYIHWVRQAPGQGLEWIG
WIDPENGDTEYAPKFQGRATMTADTSISTAYMELSRLRSDDTAVYYCNA
GGRFAYWGQGTLVTVSS,

or a polypeptide with at least 80%, 82%, 85%, 87%, 89%, 90%, 92%, 94%, 95%, 96%, 97%, 98% or 99% amino acid sequence identity thereto.

### Light Chain

```
  (SEQ ID NO: 8)
DIQMTQSPSTLSASVGDRVTITCKSSQSLLNSGNQKNYLAWYQQKPGKA
PKLLIYGASTRESGVPSRFSGSGSGTEFTLTISSLQPDDFATYYCQNDH
SYPYTFGGGTKVEIK
or
  (SEQ ID NO: 122)
DIQMTQSPSTLSASVGDRVTITCKSSQSLLNSGNQKNYLAWYQQKPGKA
PKLLIYGASTRESGVPSRFSGSGSGTEFTLTISSLQPDDFATYYCQNDH
SYPYTFGGGTKVEIKENLYFQGAAALE,
```

or a polypeptide with at least 80%, 82%, 85%, 87%, 89%, 90%, 92%, 94%, 95%, 96%, 97%, 98% or 99% amino acid sequence identity thereto.

### Exemplary Pharmaceutical Compositions and Delivery

The disclosure provides a composition comprising, consisting essentially of, or consisting of the above-described antibody, antibody fragment, such as a single chain polypeptide, polypeptide, or gene transfer vector and a pharmaceutically acceptable (e.g., physiologically acceptable) carrier, or an antibody or antigen binding fragment, polypeptide, or gene transfer vector thereof optionally with a pharmaceutically acceptable (e.g., physiologically acceptable) carrier. When the composition consists essentially of the antibody, antibody fragment, e.g., single chain polypeptide, polypeptide, or gene transfer vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the gene transfer vector and the pharmaceutically acceptable carrier, or the antibody, antigen binding fragment thereof or polypeptide optionally with a pharmaceutically acceptable carrier, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene transfer vector or an antibody or antigen binding fragment thereof or polypeptide described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene transfer vector, antibody or antigen binding fragment thereof or polypeptide is administered in a composition formulated to protect the gene transfer vector or antibody or antigen binding fragment thereof or polyepeptide from damage prior to administration. For example, the composition can be formulated to reduce loss of the gene transfer vector, antibody or fragment thereof or polypeptide on devices used to prepare, store, or administer the gene transfer vector, such as glassware, syringes, or needles. The composition can be formulated to decrease the light sensitivity and/or temperature sensitivity of the gene transfer vector or an antibody or antigen binding fragment thereof. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the method. Formulations for gene transfer vector-containing compositions are further described in, for example, Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the gene transfer vector or antibody or antigen binding fragment thereof or polypeptide can be present in a composition with other therapeutic or biologically-active agents. For example, factors that control inflammation, such as ibuprofen or steroids, can be part of the composition to reduce swelling and inflammation associated with in vivo administration of the gene transfer vector or the antibody or antigen binding fragment thereof or polypeptide. Immune system stimulators or adjuvants, e.g., interleukins, lipopolysaccharide, and double-stranded RNA, can be administered to enhance, diminish or modify the anti-CCKBR immune response. Antibiotics, i.e., microbicides and fungicides, can be present to treat existing infection and/or reduce the risk of future infection, such as infection associated with gene transfer procedures.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho) esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the gene transfer vector, antibody or antigen binding fragment thereof. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the gene transfer vectors, antibody or antigen binding fragment thereof or polypeptide, may be intracerebral (including but not limited to intraparenchymal, intraventricular, or intracisternal), intrathecal (including but not limited to lumbar or cisterna magna), or systemic, including but not limited to intravenous, oral, or any combination thereof, using devices known in the art. Delivery may also be via surgical implantation of an implanted device.

The dose of the active agent in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the gene transfer vector, antibody or antigen binding fragment thereof or polypeptide described herein. A “therapeutically effective amount” refers to an amount effective, at dosages and for periods of time necessary, to achieve a desired therapeutic result. The therapeutically effective amount may vary according to factors such as the extent of pathology, age, sex, and weight of the individual, and the ability of the gene transfer vector, antibody or antigen binding fragment thereof to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. A therapeutically effective amount may be between 1×10¹⁰ genome copies to 1×10¹³ genome copies. A therapeutically effective amount may be between 1×10¹² genome copies to 1×10¹⁵ genome copies (total). A therapeutically effective amount may be between 1×10¹² genome copies/kg to 1×10¹⁵ genome copies/kg.

The dose of antibody or antigen binding fragment thereof or polypeptide in the composition required to achieve a particular therapeutic effect typically is administered in units of antibody or antigen binding fragment or polypeptide per kg (mg/kg) or total dose (mg). One of ordinary skill in the art can readily determine an appropriate dose range to treat a patient having a particular disease or disorder, based on these and other factors that are well known in the art. A therapeutically effective amount of antibody or antigen binding fragment or polypeptide thereof may be between 25 to 200 mg, e.g., 50 to 100 mg, 25 to 50 mg, 50 to 75 mg, 100 to 150 mg, 150 to 200 mg, 200 mg to 300 mg, 300 mg to 400 mg, 400 mg to 500 mg, or 500 mg to 600 mg.

A therapeutically effective amount of antibody or antigen binding fragment thereof or polypeptide may be between 1 mg/kg to 20 mg/kg, e.g., 2 to 5 mg/kg, 5 to 7 mg/kg or 10 to 15 mg/kg.

In one embodiment, the composition is administered once to the mammal. It is believed that a single administration of the composition will result in persistent expression of the anti-CCKBR antibody or fragment in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of gene transfer vector comprising a nucleic acid sequence which encodes an antibody directed against CCKBR, or a therapeutically effective amount of the antibody or antigen binding fragment thereof or polypeptide as described above.

### Subjects

The subject may be any animal, including a human and non-human animal. Non-human animals include all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats. Subjects may include animals treated at veterinary clinics.

Exemplary subjects include human subjects suffering from or at risk for the medical diseases and conditions described herein. The subject is generally diagnosed with the condition of the subject disclosure by skilled artisans, such as a medical practitioner.

The methods of the disclosure described herein can be employed for subjects of any species, gender, age, ethnic population, or genotype. Accordingly, the term subject includes males and females, and it includes elderly, elderly-to-adult transition age and adult age subjects, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the disclosure may be more appropriate for some ethnic populations such as Caucasians, especially northern European populations, as well as Asian populations.

The term subject also includes subjects of any genotype or phenotype as long as they are in need of the disclosure, as described above. In addition, the subject can have the genotype or phenotype for any hair color, eye color, skin color or any combination thereof. The term subject includes a subject of any body height, body weight, or any organ or body part size or shape.

### Exemplary Embodiments

In one embodiment, a composition is provided comprising a humanized anti-human CCKBR antibody, or an antigen binding fragment thereof, or a polypeptide, that inhibits human CCKBR activity, wherein the antibody, the antigen binding fragment thereof, or the polypeptide has: i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO:33); and/or
ii) a variable region comprising a first CDR comprising QSLLNSGNQKNY (SEQ ID NO:34) operably linked to a second CDR comprising GAS operably linked to a third CDR comprising QNDHSYPYT (SEQ ID NO:36). In one embodiment, the antibody is a scFv. In one embodiment, a the first, second, or third CDR, or any combination thereof, of variable region i) is/are flanked by one or more human Ig framework sequences.

In one embodiment, one or more human Ig framework sequences comprise QVQLVQSGAEVKKPGASVKVSCKAS (SEQ ID NO:51), IHWVRQAPGQGLEWIG (SEQ ID NO:52), EYAPKFQGRATMTADTSISTAYMELSRLRSDDTAVYYC (SEQ ID NO:53), WGQGTLVTVSS (SEQ ID NO:54), QVQLVQSGAEVKKPGASVKVSCKAS (SEQ ID NO:59), IHWVRQATGQGLEWMGW (SEQ ID NO:60), EYAQKFQGRVTMTRDTSINTAYMELSSLRSEDTAVYYC (SEQ ID NO: 61), WGQGTLVTVSS (SEQ ID NO:62), QVQLVQSGAEVKKPGASVKVSCKAS (SEQ ID NO:67), IHWVRQATGQGLEWMGW (SEQ ID NO:68), EYAQKFQGRVTMTRDTSINTAYMELSSLRSEDTAVYYC (SEQ ID NO:69), WGQGTLVTVSS (SEQ ID NO:70), QVQLVQSGAEVKKPGASVKVSCKAS (SEQ ID NO:75), IHWVRQAPGQGLEWIGW (SEQ ID NO:76), YAPKFQGRATMTADTSISTAYMELSRLRSDDTAVYYC (SEQ ID NO:77), WGQGTLVTVSS (SEQ ID NO:78), DIQMTQSPSTLSASVGDRVTITCKSS (SEQ ID NO:79), LAWYQQKPGKAPKLLIY (SEQ ID NO:80), TRESGVPSRFSGSGSGTEFTLTISSLQPDDFATYYC (SEQ ID NO:81), FGGGTKVEIK (SEQ ID NO:82), DIQMTQSPSSLSASVGDRVTITCKSS (SEQ ID NO:100), LAWYQQKPGKAPKLLIY (SEQ ID NO:101), TRESGVPSRFSGSGSGTDFTLTISSLQPEDFATYYC (SEQ ID NO:102), FGGGTKLEIKENLYFQGAAALE (SEQ ID NO:103), MAQVQLVQSGAEVKKPGASVKVSCKAS (SEQ ID NO:104), IHWVRQAPGQGLEWIGW (SEQ ID NO:105), EYAPKFQGRATMTADTSISTAYMELSRLRSDDTAVYYC (SEQ ID NO: 106), WGQGTLVTVSS (SEQ ID NO:107), DIQMTQSPSTLSASVGDRVTITCKSS (SEQ ID NO:108), LAWYQQKPGKAPKLLIY (SEQ ID NO:109), TRESGVPSRFSGSGSGTEFTLTISSLQPDDFATYYC (SEQ ID NO:110), FGGGTKVEIKENLYFQGAAALE (SEQ ID NO:111), or a polypeptide with at least 80%, 85%, 90%, 92%, 94%, 95%, 96%, 97%, 98%, or 99% amino acid sequence identity thereto. In one embodiment, a the first, second, or third CDR, or any combination thereof, of variable region ii) is/are flanked by human Ig framework sequences. In one embodiment, the one or more human Ig framework sequences comprise DIQMTQSPSSLSASVGDRVTITCKSS (SEQ ID NO:55), LAWYQQKPGKAPKLLI (SEQ ID NO:56), TRESGVPSRFSGSGSGTDFTLTISSLQPEDFATYYC (SEQ ID NO:57), FGGGTKLEIK (SEQ ID NO:58), DIQMTQSPSSLSASVGDRVTITCRAS (SEQ ID NO:63), LAWYQQKPGKVPKLLIY (SEQ ID NO:64), TRESGVPSRFSYSGSGTDFTLTISSLQPEDVWTYYC (SEQ ID NO:65), FGQGTKLEIK (SEQ ID NO:66), DIVMTQSPDSLAVSLGERATINCKS (SEQ ID NO: 71), LAWYQQKPGQPWKLLIY (SEQ ID NO:72), TRESGVPDRFSGSGSGTDFTLTISSLQAEDVYVYY (SEQ ID NO:73), FFQGTKVEIK (SEQ ID NO:74), DIQMTQSPSTLSASVGDRVTITCKSS (SEQ ID NO:79), LAWYQQKPGKAPKLLIY (SEQ ID NO:80), TRESGVPSRFSGSGSGTEFTLTISSLQPDDFATYYC (SEQ ID NO:81), FGGGTKVEIK (SEQ ID NO:82), DIQMTQSPSSLSASVGDRVTITCKSS (SEQ ID NO:100), LAWYQQKPGKAPKLLIY (SEQ ID NO:101), TRESGVPSRFSGSGSGTDFTLTISSLQPEDFATYYC (SEQ ID NO:102), FGGGTKLEIKENLYFQGAAALE (SEQ ID NO:103), MAQVQLVQSGAEVKKPGASVKVSCKAS (SEQ ID NO:104), IHWVRQAPGQGLEWIGW (SEQ ID NO:105), EYAPKFQGRATMTADTSISTAYMELSRLRSDDTAVYYC (SEQ ID NO: 106), WGQGTLVTVSS (SEQ ID NO: 107), DIQMTQSPSTLSASVGDRVTITCKSS (SEQ ID NO:108), LAWYQQKPGKAPKLLIY (SEQ ID NO:109), TRESGVPSRFSGSGSGTEFTLTISSLQPDDFATYYC (SEQ ID NO:110), FGGGTKVEIKENLYFQGAAALE (SEQ ID NO:111), or a polypeptide with at least 80%, 85%, 90%, 92%, 94%, 95%, 96%, 97%, 98%, or 99% amino acid sequence identity thereto. In one embodiment, one or more human Ig framework sequences comprise MAQVQLVQSGAEVKKPGASVKVSCKAS (SEQ ID NO:123); IHWVRQAPGQGLEWIGW (SEQ ID NO:124); EYAPKFQGRATMTADTSISTAYMELSRLRSDDTAVYYC (SEQ ID NO: 125); WGQGTLVTV (SEQ ID NO:126), or a polypeptide with at least 80%, 85%, 90%, 92%, 94%, 95%, 96%, 97%, 98%, or 99% amino acid sequence identity thereto. In one embodiment, one or more human Ig framework sequences comprise DIQMTQSPSSLSASVGDRVTITCKSS (SEQ ID NO127); LAWYQQKPGKAPKLLIY (SEQ ID NO;128); TRESGVPSRFSGSGSGTDFTLTISSLQPEDFATYYC (SEQ ID NO:129); FGGGTKLEIKENLYFQGAAALE (SEQ ID NO:130); DIQMTQSPSTLSASVGDRVTITCKSS (SEQ ID NO:131); LAWYQQKPGKAPKLLIY (SEQ ID NO:132); TRESGVPSRFSGSGSGTEFTLTISSLQPDDFATYYC (SEQ ID NO:133); FGGGTKVEIKENLYFQGAAALE (SEQ ID NO:134), or a polypeptide with at least 80%, 85%, 90%, 92%, 94%, 95%, 96%, 97%, 98%, or 99% amino acid sequence identity thereto. In one embodiment, the composition further comprises a pharmaceutically acceptable carrier. In one embodiment, a the variable region i) and the variable region ii) are joined together by a linker. In one embodiment, the composition comprises the antigen binding fragment thereof.

In one embodiment, an isolated cell is provided comprising an expression cassette comprising a promoter operably linked to nucleic acid sequences encoding a humanized anti-human CCKBR antibody, or an antigen binding fragment thereof, or a polypeptide, that inhibits human CCKBR activity, wherein the antibody, the antigen binding fragment thereof, or the polypeptide has: i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO:33); and/or ii) a variable region comprising a first CDR comprising QSLLNSGNQKNY (SEQ ID NO:34) operably linked to a second CDR comprising GAS operably linked to a third CDR comprising QNDHSYPYT (SEQ ID NO:36). In one embodiment, the cells is a mammalian cell. In one embodiment, the cell is a primate cell. In one embodiment, the cell is a human cell. In one embodiment, the promoter is a heterologous promoter.

In one embodiment, an isolated nucleic acid is provided comprising a promoter operably linked to a nucleotide sequence which encodes at least the variable region of a human heavy or light chain that binds human CCKBR, wherein the chain comprises: i) a variable region comprising a first complementarity determining region (CDR) comprising GFNIKDYY (SEQ ID NO: 31) operably linked to a second CDR comprising IDPENGDT (SEQ ID NO: 32) operably linked to a third CDR comprising NAGGRFAY (SEQ ID NO: 33); and/or ii) a variable region comprising a first CDR comprising QSLLNSGNQKNY (SEQ ID NO:34) operably linked to a second CDR comprising GAS operably linked to a third CDR comprising QNDHSYPYT (SEQ ID NO:36). In one embodiment, the isolated nucleic acid is on a vector. In one embodiment, the isolated nucleic acid is a viral vector.

In one embodiment, an isolated polypeptide is provided comprising QVQLVQSGAEVKKPGASVKVSCKASGFNIKDYYIHWVRQAPGQGLEWI GWIDPENGDTEYAPKFQGRATMTADTSISTAYMELSRLRSDDTAVYYC NAGGRFAYWGQGTLVTVSS (SEQ ID NO:1), PKLLIYGASTRESGVPSRFSGSGSGTDFTLTISSLQPEDFATYYCQNDHSY PYTFGGGTKLEIK (SEQ ID NO:2), QVQLVQSGAEVKKPGASVKVSCKASGFNIKDYYIHWVRQATGQGLEW MGWIDPENGDTEYAQKFQGRVTMTRDTSINTAYMELSSLRSEDTAVYY CNAGGRFAYWGQGTLVTVSS (SEQ ID NO:3), DIQMTQSPSSLSASVGDRVTITCRASQSLLNSGNQKNYLAWYQQKPGKV PKLLIYGASTRESGVPSRFSYSGSGTDFTLTISSLQPEDVWTYYCQNDHS YPYTFGQGTKLEIK (SEQ ID NO:4), QVQLVQSGAEVKKPGASVKVSCKASGFNIKDYYIHWVRQATGQGLEW MGWIDPENGDTEYAQKFQGRVTMTRDTSINTAYMELSSLRSEDTAVYY CNAGGRFAYWGQGTLVTVSS (SEQ ID NO:5), DIVMTQSPDSLAVSLGERATINCKSSQSLLNSGNQKNYLAWYQQKPGQP WKLLIYGASTRESGVPDRFSGSGSGTDFTLTISSLQAEDVYVYYCONDHS YPYTFFQGTKVEIK (SEQ ID NO:6), QVQLVQSGAEVKKPGASVKVSCKASGFNIKDYYIHWVRQAPGQGLEWI GWIDPENGDTEYAPKFQGRATMTADTSISTAYMELSRLRSDDTAVYYC NAGGRFAYWGQGTLVTVSS (SEQ ID NO:7), DIQMTQSPSTLSASVGDRVTITCKSSQSLLNSGNQKNYLAWYQQKPGKA PKLLIYGASTRESGVPSRFSGSGSGTEFTLTISSLQPDDFATYYCQNDHSY PYTFGGGTKVEIK (SEQ ID NO:8), MAQVQLVQSGAEVKKPGASVKVSCKASGFNIKDYYIHWVRQAPGQGL EWIGWIDPENGDTEYAPKFQGRATMTADTSISTAYMELSRLRSDDTAVY YCNAGGRFAYWGQGTLVTV (SEQ ID NO:140), DIQMTQSPSSLSASVGDRVTITCKSSQSLLNSGNQKNYLAWYQQKPGKA PKLLIYGASTRESGVPSRFSGSGSGTDFTLTISSLQPEDFATYYCQNDHSY PYTFGGGTKLEIKENLYFQGAAALE (SEQ ID NO: 141), DIQMTQSPSTLSASVGDRVTITCKSSQSLLNSGNQKNYLAWYQ QKPGKAPKLLIYGASTRESGVPSRFSGSGSGTEFTLTISSLQPDDFATYYC QNDHSYPYTFGGGTKVEIKENLYFQGAAALE (SEQ ID NO:142), MAQVQLVQSGAEVKKPGASVKVSCKASGFNIKDYYIHWVRQAPGQGL EWIGWIDPENGDTEYAPKFQGRATMTADTSISTAYMELSRLRSDDTAVY YCNAGGRFAYWGQGTLVTV (SEQ ID NO:120), PKLLIYGASTRESGVPSRFSGSGSGTDFTLTISSLQPEDFATYYCQNDHSY PYTFGGGTKLEIKENLYFQGAAALE (SEQ ID NO:121), DIQMTQSPSTLSASVGDRVTITCKSSQSLLNSGNQKNYLAWYQQKPGKA PKLLIYGASTRESGVPSRFSGSGSGTEFTLTISSLQPDDFATYYCQNDHSY PYTFGGGTKVEIKENLYFQGAAALE (SEQ ID NO:122), or an amino acid sequence having 1 to 5 amino acid substitutions in one or more of the framework regions, or an antigen binding portion thereof.

In one embodiment, a method to inhibit or treat depression or anxiety in a mammal is provided comprising: administering to a mammal a composition comprising an effective amount of a nucleotide sequence which encodes at least the variable region of a human heavy or light chain that binds human CCKBR, wherein the chain comprises: i) a variable region comprising a first complementarity determining region (CDR) comprising GFNIKDYY (SEQ ID NO: 31) operably linked to a second CDR comprising IDPENGDT (SEQ ID NO: 32) operably linked to a third CDR comprising NAGGRFAY (SEQ ID NO: 33); and/or ii) a variable region comprising a first CDR comprising QSLLNSGNQKNY (SEQ ID NO:34) operably linked to a second CDR comprising GAS operably linked to a third CDR comprising QNDHSYPYT (SEQ ID NO:36), or an antibody or fragment comprising i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO:33); and/or ii) a variable region comprising a first CDR comprising QSLLNSGNQKNY (SEQ ID NO:34) operably linked to a second CDR comprising GAS operably linked to a third CDR comprising QNDHSYPYT (SEQ ID NO:36). In one embodiment, the heavy chain is an IgG heavy chain. In one embodiment, the light chain is an Igk light chain. In one embodiment, the antibody fragment is administered. In one embodiment, the fragment is Fab′ or scFv. In one embodiment, the mammal is a human. In one embodiment, the composition is systemically administered. In one embodiment, the composition is injected. In one embodiment, wherein the nucleotide sequence is in a viral vector. In one embodiment, the composition inhibits dorsal root ganglion (DRG) neurons.

In one embodiment, a method to inhibit or treat pain in a mammal is provided comprising: administering to a mammal a composition comprising an effective amount of a nucleotide sequence which encodes at least the variable region of a human heavy or light chain that binds human CCKBR, wherein the chain comprises: i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO:33); and/or ii) a variable region comprising a first CDR comprising QSLLNSGNQKNY (SEQ ID NO: 34) operably linked to a second CDR comprising GAS operably linked to a third CDR comprising QNDHSYPYT (SEQ ID NO:36), or an antibody or fragment comprising i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO:33); and/or ii) a variable region comprising a first CDR comprising QSLLNSGNQKNY (SEQ ID NO: 34) operably linked to a second CDR comprising GAS operably linked to a third CDR comprising QNDHSYPYT (SEQ ID NO:36). In one embodiment, the mammal has neuropathic pain. In one embodiment, the mammal was exposed to blunt force trauma. In one embodiment, the mammal has traumatic brain injury. In one embodiment, the mammal is a human. In one embodiment, the composition is systemically administered. In one embodiment, the composition is injected. In one embodiment, wherein the nucleotide sequence is in a viral vector. In one embodiment, the composition inhibits dorsal root ganglion (DRG) neurons.

The invention will be further described by the following non-limiting examples.

### EXAMPLE 1

### Exemplary Overview of Generation of Humanized and CDR Modified/scFv Antibodies

Design humanized variants (3VH×3VL) per humanization
1 parental sequence
   3 variable heavy chains, 3 variable light chains in total
Construct nine humanized scFvs (each combination of three designed heavy chains and three designed light chains) as well as produced additional stock of parental scFv using E. coli or CHO Transient Production for ongoing assay development
Affinity measurement of 6 constructed humanized scFv variants against human peptide with binding affinity in the nanomolar range for efficacy testing. targeting CCKBR based on affinity of the parent antibody
Assess humanized variants by T20 humanness score compared to parental antibody.
Humanization processes yielding humanized scFv
DNA scale-up (10 scFvs), 0.03 L transient production in CHO (14 day process) or E. coli cells and Protein L/affinity purification QC DNA sequence confirmation
CDR3 modified/scFv antibodies.
Assess expression levels
   Demonstration of Humanization and Characterization of CCKBR Single Chain Fragment Variable Antibody (ₕᵤₘₐₙsCFv)

Any of HCDR1-3 and/or LCDR1-3, which are structurally related to SEQ ID Nos. 1-8, respectively, may be employed in the antibodies or fragments thereof

Humanization of CCKBR scFv

Variable heavy (VH) and light (VL) regions of the mscFv77-2 parental are sequenced and compared using BLASTp (NCBI) to identify homology with known human VH and VL antibody sequences. The three most homologous candidates to the murine sequences are identified taking into account framework homology, maintenance of key framework residues, and canonical loop structure (based on a combined IMGT/Kabat CDR labelling approach). The three humanized VH domains and three humanized VL domains (designed 9 humanized variants (3VH×3VL)) are synthesized and cloned into the pET32a expression vector. Resultant recombinant chimeric antibodies are expressed, purified from E. coli Rosetta-gami cytoplasm and assessed by ELISA for kinetic interaction analyses. Yields of 3 to 5 mg/l of pure, soluble, active scFv fragments are obtained from shake flask cultures.

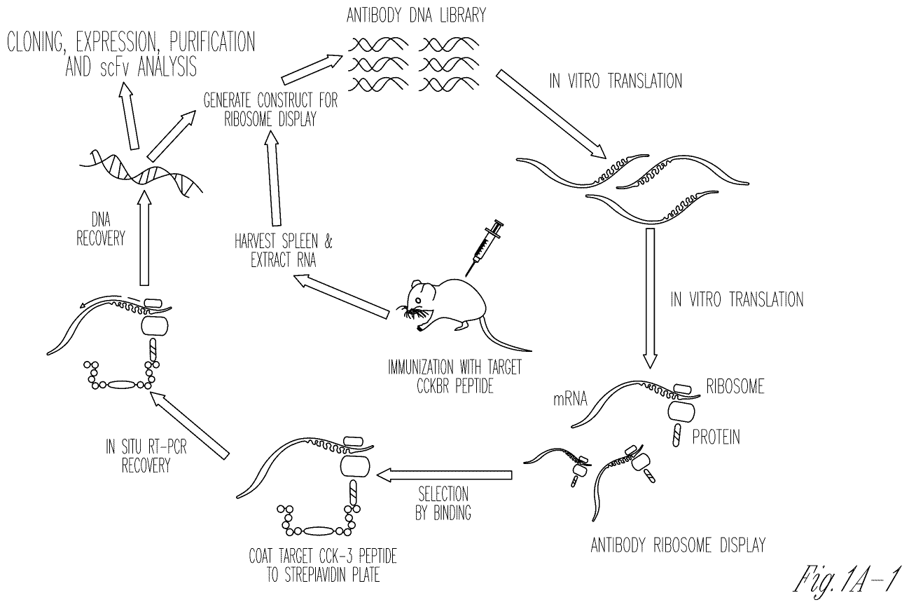

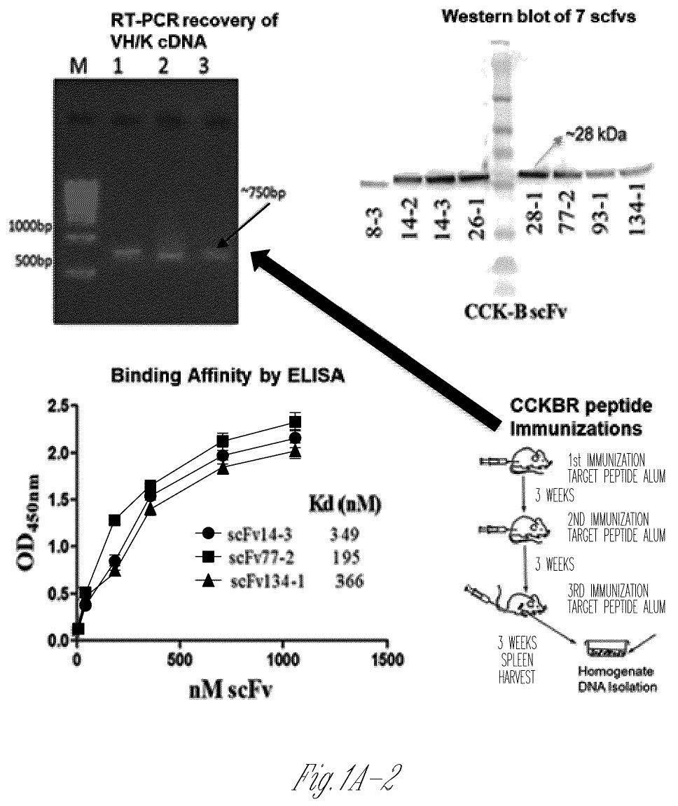

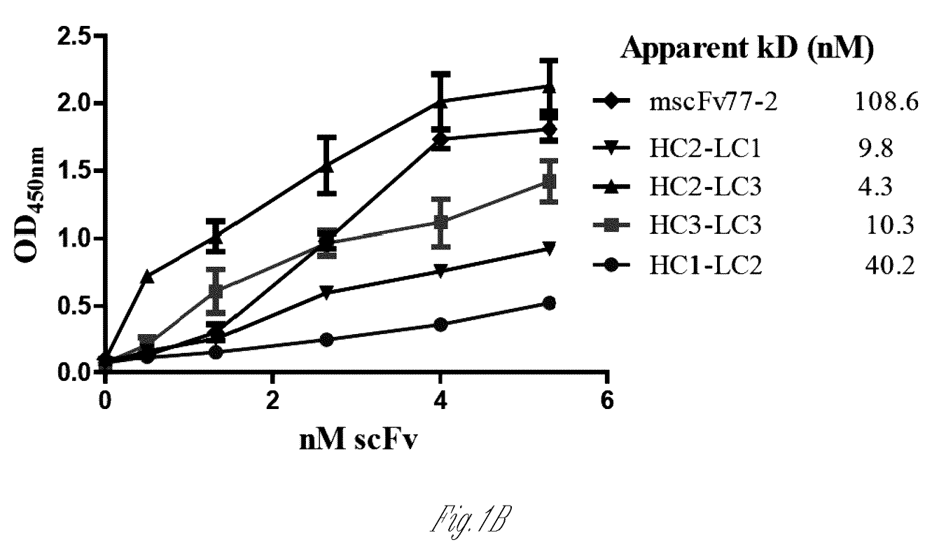

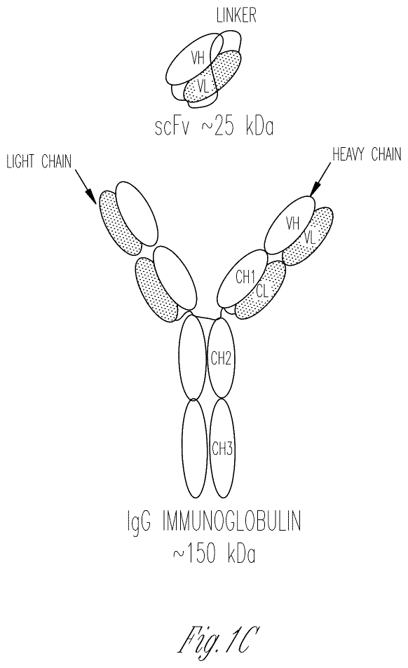

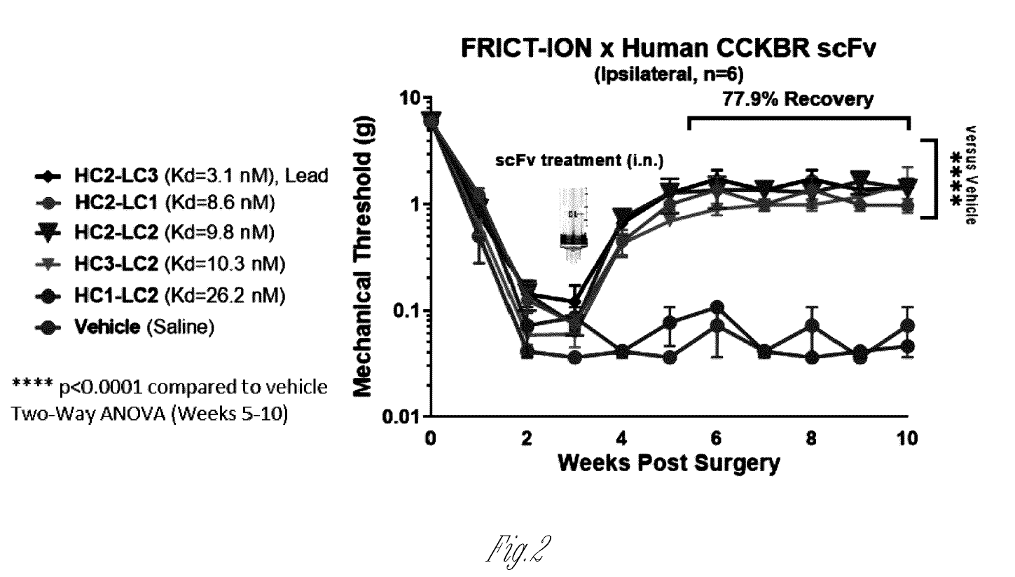

To determine whether the humanized scFvs could bind specifically to their corresponding mouse and human CCKAR peptides, the soluble, purified scFv antibodies are assessed by ELISA assays. The humanized CCKAR scFv reacted only with mouse and human peptides, whereas the negative control anti-CCKAR antibody did not react. The promising scFv clones identified from indirect ELISA respectively bind to human CCKBR peptide with desirable dissociation constants compared to parental mscFv77-2. Among them, certain clones exhibit the highest binding affinity by achieving more than a 25-fold affinity improvement. One clone in particular results in over a 50-fold enhancement compared with the parental mscFv77-2 sequence (FIG. 1B).

Immunogenicity Prediction of Humanized scFv Variants

The predicted immunogenicity using the T20 score of our humanized variants (CDR grafted) is anticipated to be very low (Table 1). T20 score is used to measure the “humanness” of monoclonal antibody variable region sequences. This scoring system was developed by Gao et al [Monoclonal antibody humanness score and its applications. 2013. BMC Biotechnology, 13: 55], using a database of over 38,000 human antibody sequences. In this method, a protein BLAST of this database is performed and the test humanized Ab is compared against these human sequences. The humanized antibody is compared to the top 20 human Ab BLAST matches and scored for similarity to these sequences. The highest possible score is 100 (most human-like).

Validation of this method was shown when Gao et al tested this immunogenicity scoring method on more than 90 antibodies that have been used clinically in humans, and found that antibodies with T20 scores of FR & CDR sequences above 80 were not immunogenic while T20 scores for FR sequences only, which were above 85 were not immunogenic. Using the T20 values of the current humanized scFv77-2 variants, very low immunogenicity is expected in patients (Table 1). This shows that the CDR-grafted antibodies are more human and expected to be less immunogenic than the chimeric (parental), that all the hscFv77-2 variants are expected to have low immunogenicity.

TABLE 1
T20 Humanness Assessment for hscFv77-2 variants

| | | T20 | T20 |
|---|---|---|---|
| | | Analyzer | Analyzer |
| | | Score | Score |
| | | (Full | (Framework |
| Name | Species | Length) | only) |
| mscFv77-2HC_Parental | Mus musculus | 62.9 | 65.7 |
| hu scFv77-2.HC1 | Homo sapiens | 80.1 | 87.6 |
| hu scFv77-2.HC2 | Homo sapiens | 80.0 | 88.0 |
| hu scFv77-2.HC3 | Homo sapiens | 80.1 | 90.0 |
| mscFv77-2-LC Parental | Mus musculus | 80.9 | 86.3 |
| hu scFv77-2.LC1) | Homo sapiens | 89.8 | 98.6 |
| hu scFv77-2.LC2 | Homo sapiens | 80.5 | 98.0 |
| hu scFv77-2.LC3 | Homo sapiens | 81.2 | 96.0 |

The humanness scores for the parental and humanized antibodies are shown in the table above. Based on the method, a score of 85 or above is indicative of a human-like heavy chain framework, and a score of 90 or above is indicative of humanness for a kappa light chain framework. For full-length variable regions, cutoffs of 80 for the VH and 85 for the VK are recommended. T20 scores below the recommended values are indicated in red.
   Humanized CCKBR scFv Efficacy
Humanized CCKBR scFv (4 mg/kg, i.p.) is given to mice with FRICT-ION trigeminal nerve injury and the effect on mechanical hypersensitivity tested with von Frey filaments. The ₕᵤₘₐₙscFv is given in week 2 after model induction. Affinity matured humanized CCKBR receptor scFv antibody testing in a nerve neuropathic pain model demonstrates the product is highly efficacious for reduction the hypersensitivity pain measure. The scFv is tested when given once in week 2-4 to assess mechanical and cold/heat hypersensitivity. The ₕᵤₘₐₙscFv is highly effective in reducing ongoing hypersensitivity after nerve injury. Full reversal to baseline may occur in week 3 after treatment (6 weeks after model induction). Testing in the subsequent weeks allows for evidence of efficacy for reducing anxiety-and depression-like behaviors comorbid with pain. Spinal Nerve Injury (SNI) and Trigeminal Nerve Injury (FRICT-ION) models are tested with humanized scFv treatments. The humanized scFv are also tested in inflammatory leg muscle and back pain models. Effectiveness was found for reduction of pain, cold, and heat measures in all test models. Preditor stress anxiety in the absence of a pain model was also demonstrated.

### Summary

Humanized CCKBR scFvs are generated and the scFvs affinity matured
Affinity matured ₕᵤₘₐₙscFv promotes best binding affinity in the nanomolar range with T20 humanness score suitable for human testing
Affinity matured ₕᵤₘₐₙscFv with the best binding affinity is successfully tested defining efficacy in a mouse nerve injury model as well for reversal of neuropathic pain behaviors
The humanized scFv allows for treatment of acute and chronic pain where currently few treatment options exist and none can reverse the effects of nerve injury
The scFv technology is a new generation of therapeutics for acute, chronic pain, and related comorbidities including anxiety, depression, and/or diminished/disrupted cognition, as well as stress anxiety in the absence of pain.
Cold hypersensitivity, established in the mouse behavioral in vivo and in vitro electrophysiological models, is a major complaint reported by patients with trigeminal nerve injury.
Gene profiling with RNAseq in the models, comparing mice with and without model induction, provides insight into the upregulation of CCKBR during chronic pain and downregulation by the CCK-BR scFv.
CCKBR ₕᵤₘₐₙscFv reduces excitability (increasing rheobase or hyperpolarizing resting membrane potential) of human primary dorsal root ganglia (DRG) neurons. This demonstrates direct translational potential of the scFv for effectiveness in patients with chronic pain in which the excitability of DRG neurons is increased.

### Conclusion

Ribosome display is a powerful cell-free technology and this technology is widely used to select single-chain antibody fragments against the target of choice due to reduced self-immunogenicity as well as easy and inexpensive large-scale production. This rapid method is used to quickly develop repertoires of high-affinity antibodies targeting CCKBR for the studies. The scFvs developed with ribosome display have higher affinity, superior stability and solubility. Their small size has the potential for reduced self-immunogenicity. The innovation of this project includes for the first ever demonstration of permanent reversal of chronic neuropathic pain related behaviors by single dose administration of humanized scFv.

### Methods

Detailed Method of the Generation of the Humanized scFv Antibodies Using Ribosome Display

Humanization and affinity maturation of CCKBR scFv followed from generation of a mouse parental. The parental scFv binds both the human and mouse forms of target CCKBR receptor extracellular peptide sequence. Also, it contains a kappa light chain and should bind Protein L. The goal is to identify humanized variants that bind both the human and mouse peptide targets with similar or improved affinity as compared to the parental mouse scFv. The affinities of the humanized version of the scFv to the mouse and human peptides are first determined during the humanization process, where biotinylated peptide are immobilized as ligand and the scFv may be tested as analytes.

CCKBR scFv Antibody Humanization and Affinity Maturation

The humanization process is used to design 3 VH×3 VL humanized variants for recombinant production, purification, and affinity measurement (for top 9) by Octet against human and mouse forms of the target CCKBR peptide. The top humanized candidate(s) are selected for affinity maturation.

For the affinity maturation project, a ribosome display platform is used to obtain scFv variants. Randomization of the humanized scFv CDRs, as well as a pool of kappa light chain shuffled VL sequences, are employed to generate diverse libraries for affinity maturation. The variants are expressed in the bacterial cytoplasm and tested as cytoplasmic extracts (CPE's) to assess affinity for both the human and mouse peptides. The candidates may be screened utilizing the Octet platform for kinetics analysis, though alternate platforms for kinetics analysis are available for screening. The candidates are selected for recombinant production to evaluate productivity and yield from a ProL/His Tag affinity purification. Kinetic analysis of the human and mouse peptides are performed. The scFv molecules are also assayed for purity and endotoxin requirement.

Variable heavy (VH) and light (VL) regions of the CCKBR murine antibody are sequenced, a mouse homology model of the fragment variable is created and compared using BLASTp (NCBI) to identify homology with known human VH and VL antibody sequences The three most homologous candidates to the murine sequences are identified taking into account framework homology, maintenance of key framework residues, and canonical loop structure (based on a combined IMGT/Kabat CDR labelling approach) using the Bioluminate software from Schrodinger. The three humanized VH domains and three humanized VL domains are synthesized, cloned into the pET32 expression vector and expressed in Rosetta-Gami competent cells. Resultant recombinant chimeric antibodies are purified and assessed by ELISA and Octet instrument for kinetic interaction analyses. A clone, with an equilibrium dissociation constant (KD) of <130 nM, is selected for affinity maturation.

Kunamneni et al. utilized a ribosome display platform to obtain scFv variants. Randomization of the humanized scFv CDRs, as well as a pool of kappa light chain shuffled VL sequences, are employed to generate diverse libraries for affinity maturation. The variants are expressed in the bacterial cytoplasm and tested as cytoplasmic extracts (CPE's) to assess affinity for both the human and mouse peptide. The candidates are screened utilizing the Octet platform, though alternate platforms for kinetics analysis. Top three candidates are selected for recombinant production to evaluate productivity and yield from a ProL/Histag affinity purification. Kinetic analysis of the human and mouse peptides is performed. The scFv molecules are also assayed for purity and endotoxin requirement.

### Humanization

Antibody sequence analysis and homology modeling of mAb 3D structure.
Identification of key positions supporting CDR loop structure and VH-VL interface.
Design humanized variants (3VH, 3VL) using the Bioluminate software
Assess the humanness of humanized variants by T20 humanness score analyzer
Construct humanized scFvs (each combination of designed heavy chains and designed light chains) as well as produce parental scFv using Rosetta-Gami (DE3) Production
   Histag affinity purification
   Endotoxin requirement: <1 EU/mg
Assess expression levels
Affinity measurement of top constructed humanized scFvs and (1) parental scFv against (2) antigens by Octet
Octet experiment performed using inverse assay format using streptavidin biosensor (biotinylated antigen are tested as ligand and the scFv will be tested as analyte).

### Affinity Maturation

### Starting Materials

Parental humanized scFv sequence
2 mg of biotinylated human Target CCKBR peptide
1 mg of biotinylated rat Target CCKBR peptide.
1 mg of parental humanized scFv.
1 mg of parental murine scFv
   Generation of Parental Validation and scFv Ribosome Display Library Construction
The parental mouse scFv and selected humanized scFv are generated as ribosomal constructs as reference molecules for the campaign:
   (2) Gene synthesis and molecular construction
   Validation of (2) parental scFv (mouse and humanized) expressed in CPEs to human and mouse peptide antigen by ELISA.
One library (CDR-focused and/or large size light chain combination library) is generated.

### Panning

The panning strategy includes of one panning arm using biotinylated human Target CCKBR peptide on streptavidin beads. Stringency is increased during each successive round by decreasing antigen concentration on streptavidin beads, increasing the washes, and/or changing the duration of selections. The antibody variants are selected against reducing concentrations of target antigen through three consecutive selection rounds of ribosome display.

Panned outputs from library and round showing enrichment are moved forward for screening.

### Screening and Sequencing

### Screening

Following panning, 4×96-well output clones are prepared as master plates and stored for screening. Up to 384 bacterial cytoplasmic extracts (CPEs) of the output clones are assayed by ELISA for binding to human Target CCKBR peptide, incorporating the parental humanized and murine scFv as controls.

### Sequencing

Up to 2×96 positive clones are sequenced and analyzed to identify sequence-unique clones. Sequence liability analysis is performed on top hits, including isoelectric point (pI) estimation, and identification of amino acid motifs that are sensitive to post-translational modifications (e.g. deamination, glycosylation, free cysteines).

### Secondary Screening

Up to 96 target-binding, sequence-unique clones are re-arrayed. New CPE's are generated and confirmed by ELISA against:
Biotinylated human CCKBR.
Biotinylated rat CCKBR
   Kinetic analysis of up to 36 unique clones is performed using CPEs against two antigens (human Target CCKBR and mouse or rat Target CCKBR peptide) using Bio-layer Interferometry (Octet) platform. Supplemental scFv purification from CPE's followed by Octet kinetic analysis can be also performed.
   scFv Recombinant Production, Purification & Characterization

Up to 10 scFv antibody fragments are cloned into an expression vector and recombinantly expressed using the 0.03 L CHO 14-day/Rosetta-Gami guaranteed low endotoxin process along with ProL resin/Histag affinity purification. Purified antibodies are analyzed by:
Purity Analysis by CE-SDS
Affinity determination of up to 10 purified scFv against two Ag by BLI (Octet)
   Two antigens: human Target CCKBR and rat Target CCKBR

### Computational Modeling

Computational modeling was done according to Tang & Cao (2021) using Schrödinger 2020-2 software (Schrödinger, Inc., New York, NY). An advanced computational protocol was used for determining interactions between scFv and rat CCKBR peptide involving several steps. I-TASSER analysis was used to produce three-dimensional structure model of protein molecules from amino acid sequences (Zhang, 2008; Yang et al., 2016). The predicted structural models were validated using high-resolution protein structure refinement (Zhu et al., 2014), ModRefiner (Xu & Zhang, Y, 2011), and fragment-guided molecular dynamics simulation (Zhang et al., 2011).

### Molecular Docking

The refined models were docked according to the Fast Fourier Transform (FFT)-based program PIPER (Kozakov et al., 2006). Docking results were validated using LIGPLOT (Wallace et al., 1996). An interactive map identifies interactions such as hydrogen bonds, pi-pi interaction, side-chain bond, and backbone hydrogen bonds. Ligand-protein interaction maps also were used to predict the position and the interacting amino acids of the humanized CCKBR scFv and the CCKBR protein.

### EXAMPLE 2

Estimates are that in week 6, mice have experienced pain equivalent to 8 human years and can be considered chronic (Dutta & Sengupta, 2016), making chronic neuropathic pain models ideal for testing potential non-opioid therapeutics at chronic time points. Single-chain Fragment variable antibodies (scFvs) are opening a new era of therapeutics, pharmacology, and pathophysiology research. These small antibodies (˜27 kDa) are brain penetrant and praised as having promising biotherapeutic applications for the nervous and immune systems, now recognized as interactive in chronic pain. Humanized scFvs directed to a unique extracellular peptide within the CCKBR were engineered.

Humanized CCKBR scFv (hCCKBR scFv) reversed mechanical and cold hypersensitivity in three rodent model of chronic neuropathic pain with weeks of continuing efficacy after only single intraperitoneal dose. Likewise, the single-chain Fragment variable (scFv) antibody totally prevented development of anxiety, depression, and stress behaviors, as well as restores disordered cognition typical in chronic pain and stress models.

### FRICT-ION Chronic Trigeminal Neuropathic Pain Model

The trigeminal nerve injury rodent models established in the literature for the study of trigeminal orofacial pain have been refined over the years and used by numerous labs in the field of pain research for study of the neuronal pathways and mechanisms causal in pain (Vos et al., 1994; Imamura et al., 1997; Anderson et al., 2003; Xu et al., 2008; Ma et al., 2012, Obuku et al., 2013; Ding et al., 2017). The latest refinement, development of the Foramen Rotundum Inflammatory Constriction of the Trigeminal InfraOrbital Nerve (FRICT-ION) model, is a minimally invasive variant and rapid method useful for both rats and mice (Montera and Westlund, 2020). BALB/c white mice were used since they remain cooperative through the 10 weeks of behavioral testing although C57b16 can also be used with specific KO animals. One lip of anesthetized mice is secured with cotton suture to expose the buccal-cheek crease where a tiny scalpel cut exposes trigeminal nerve roots innervating the teeth. A 3-mm section of chromic gut suture is slid along the infraorbital nerve into the foramen rotundum as it enters the skull. This rapid 5-10 min method produces hypersensitivity over the subsequent week that persists over the seven week experiment. Control mice (sham-operated) undergo the same surgical procedure without nerve manipulation. Naïve mice remain untouched.

Bioefficacy of High Affinity Anti-CCKBR scFv Antibodies on Functional Phenotypes

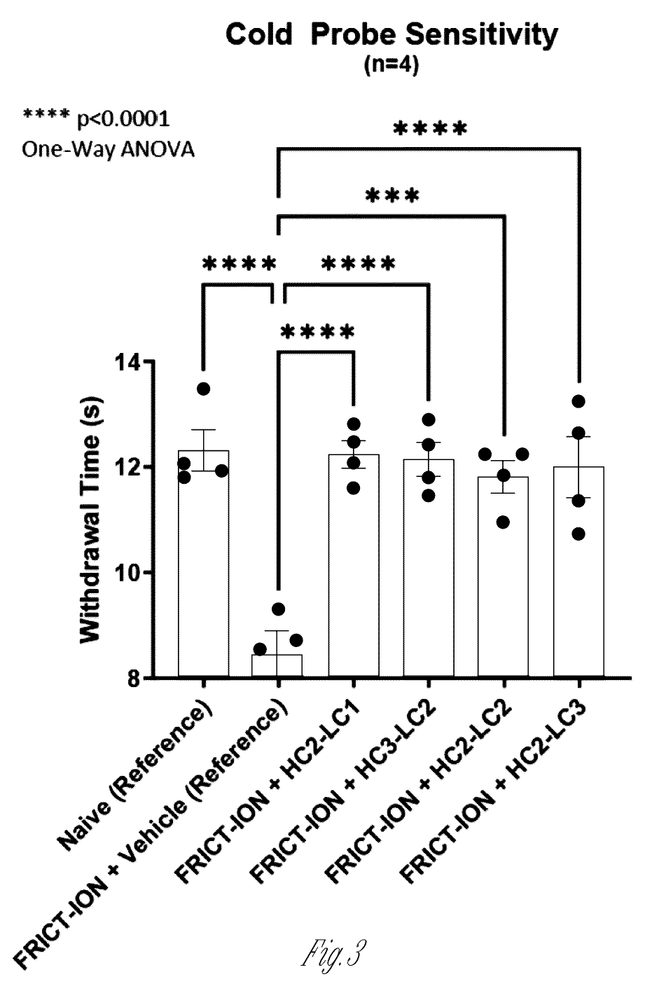

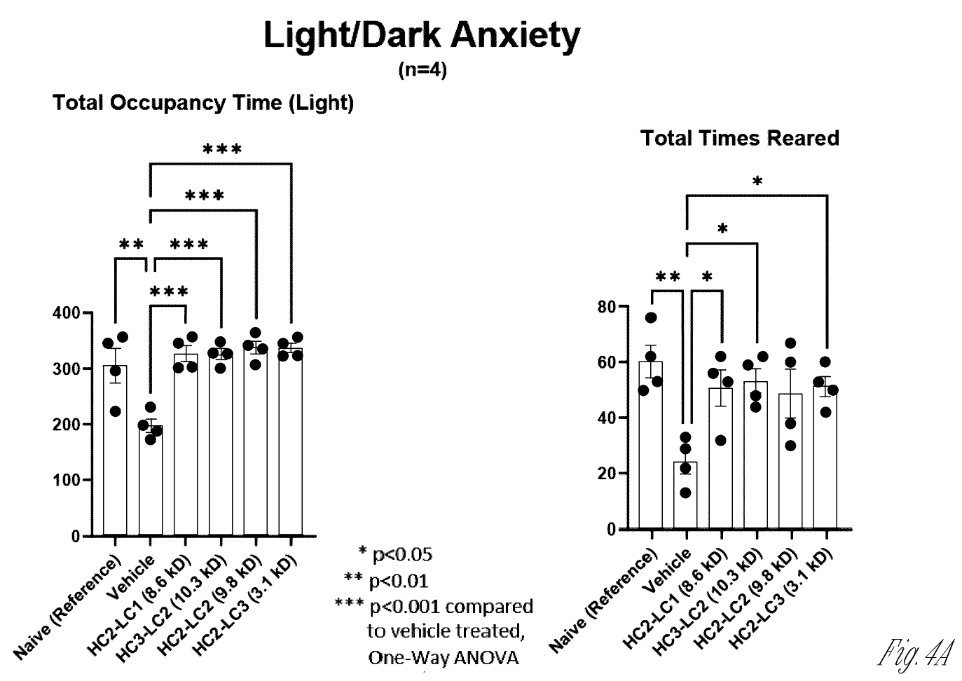

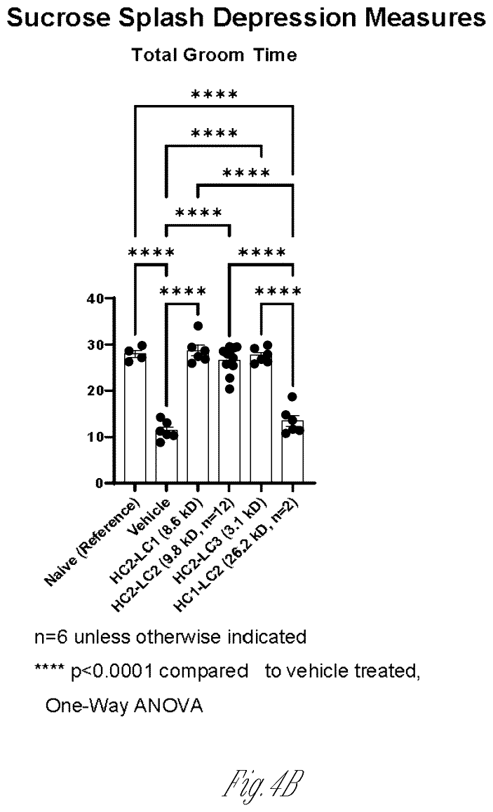

Mice with FRICT-ION chronic pain model have persistently decreased threshold for mechanical and cold stimulation in weeks 2-10 indicating hypersensitivity (allodynia, hyperalgesia) (FIGS. 2,3,5,6,7,8). The humanized CCKBR scFv HC2-LC3, HC2-LC1, HC2-LC2 were effective in reversing mechanical and cold/heat hypersensitivity back toward baseline (77.9%; 4 mg/kg, intranasal, i.n. or subcutaneous, s.c.). The HC1-LC2 variant and the vehicle were ineffective.

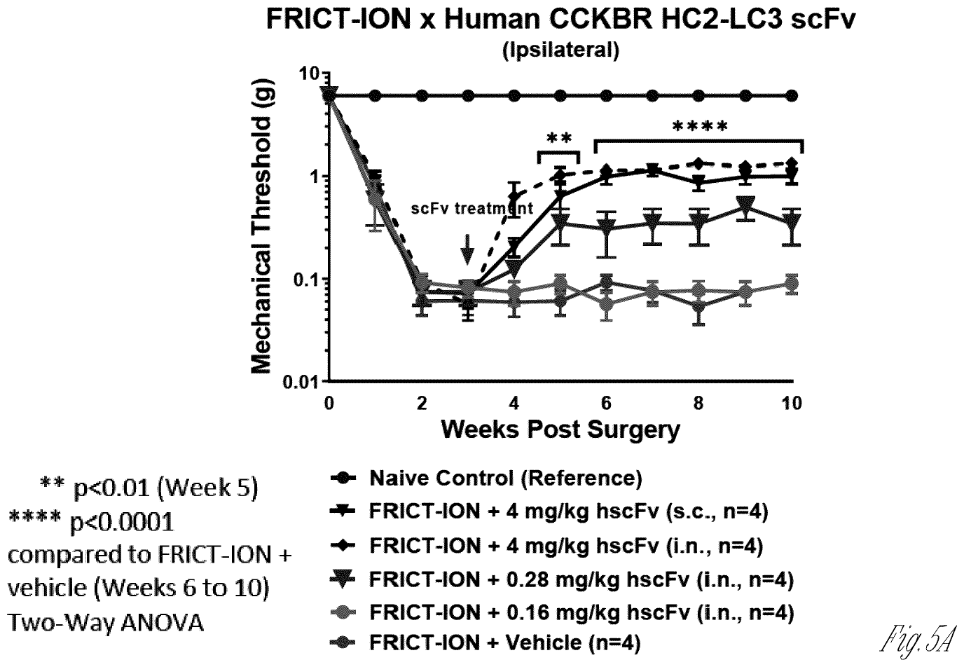

Reduction of the pain measures by treatment of FRICT-ION mice with the same humanized CCKBR scFv HC2-LC3, HC2-LC1, HC2-LC2 variants was effective in preventing the development of anxiety measures (FIGS. 4A,9). The mice given the HC1-LC2 variant or vehicle spent less time in the light and in rearing behavior indicating anxiety. Testing was done in week 7 after treatment with 4 mg/kg, i.n. Likewise, the same variants (HC2-LC3, HC2-LC1, HC2-LC2) were effective in preventing depression-like behavior (less grooming time) in treated mice with FRICT-ION, while the mice given HC1-LC2 variant and vehicle did not groom as effectively after a splash of sucrose (FIG. 4B).

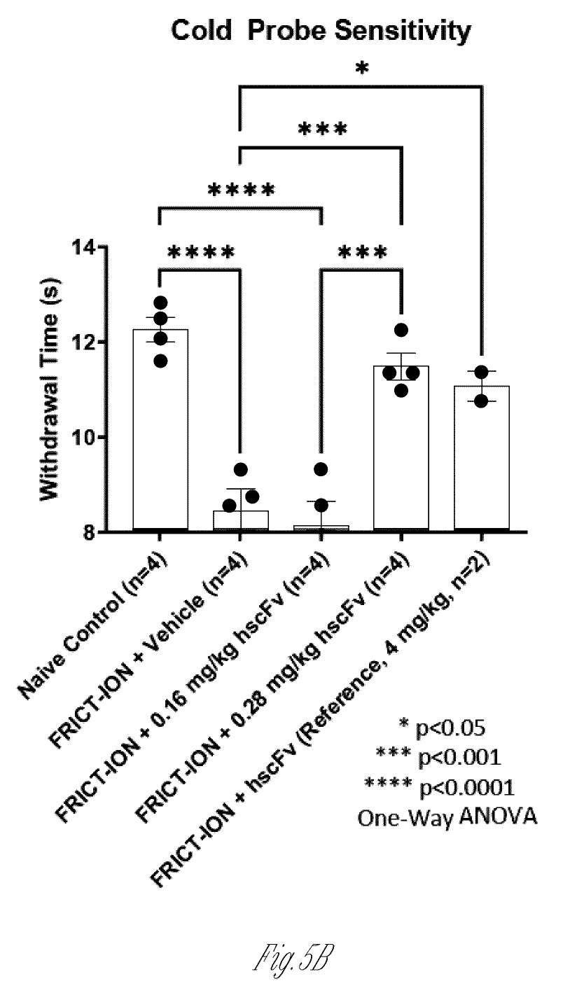

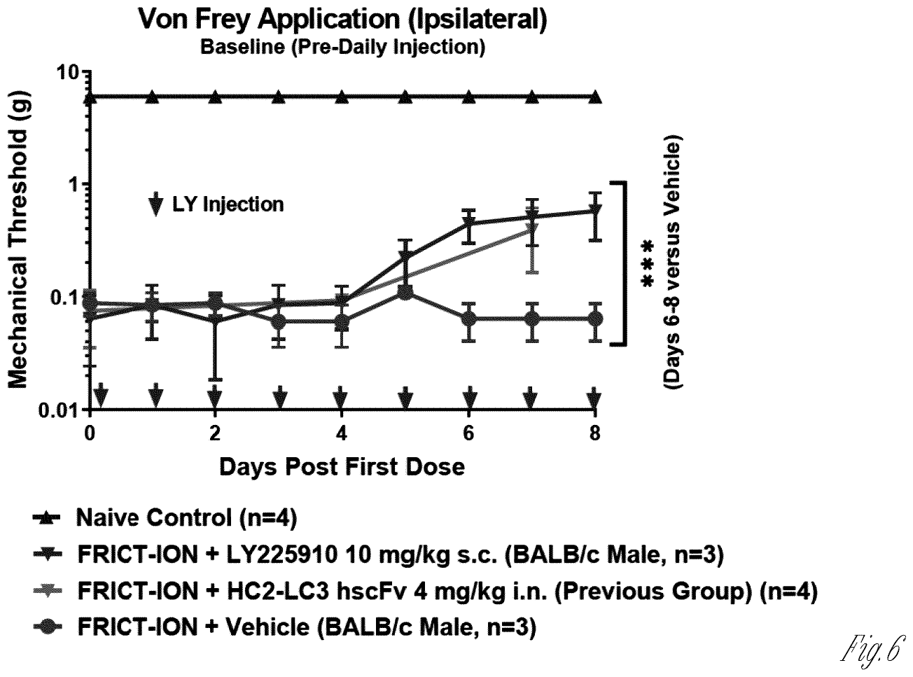

Dose Response Efficacy of Variant HC2-LC3 hscFv
Dose response (0.16, 0.28, 0.4 mg/kg) efficacy of the humanized CCKBR scFv HC2-LC3 was assessed mice with FRICT-ION craniofacial pain. The scFv antibody optimal dose or vehicle was administered locally (s.c.) or intranasally (i.n). Only the higher 0.28 and 4.0 mg/kg doses were effective in reducing the mechanical and cold pain behavior (FIG. 5). Both intranasal and local treatments were effective when 4 mg/kg HC2-LC3 was tested.

### Comparison to Commercial CCKBR Inhibitor, LY225910

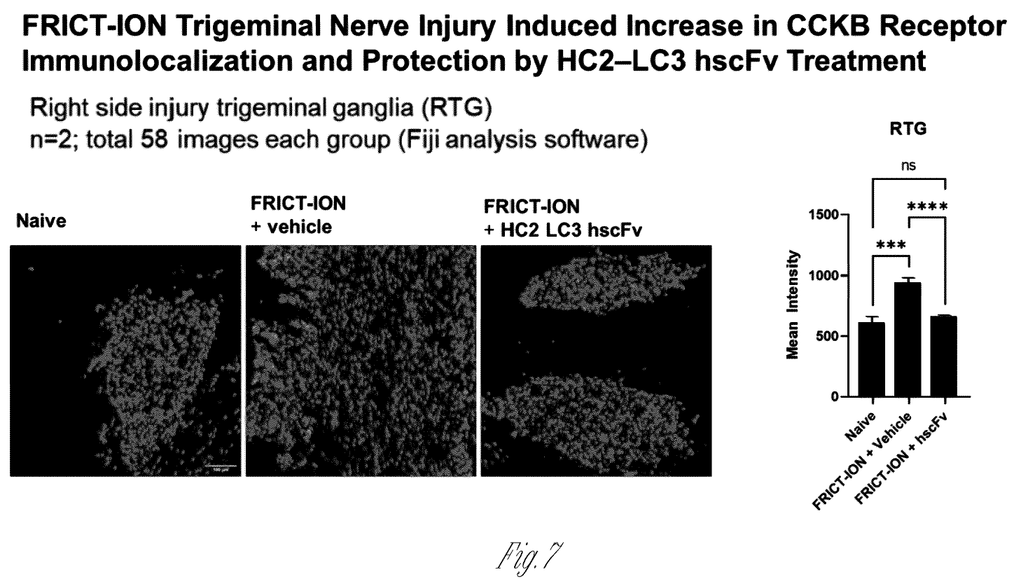

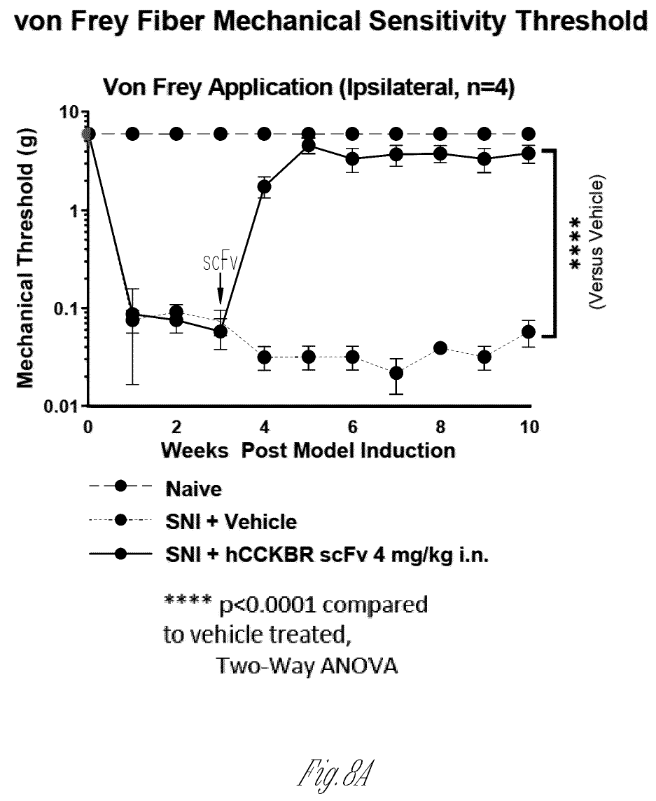

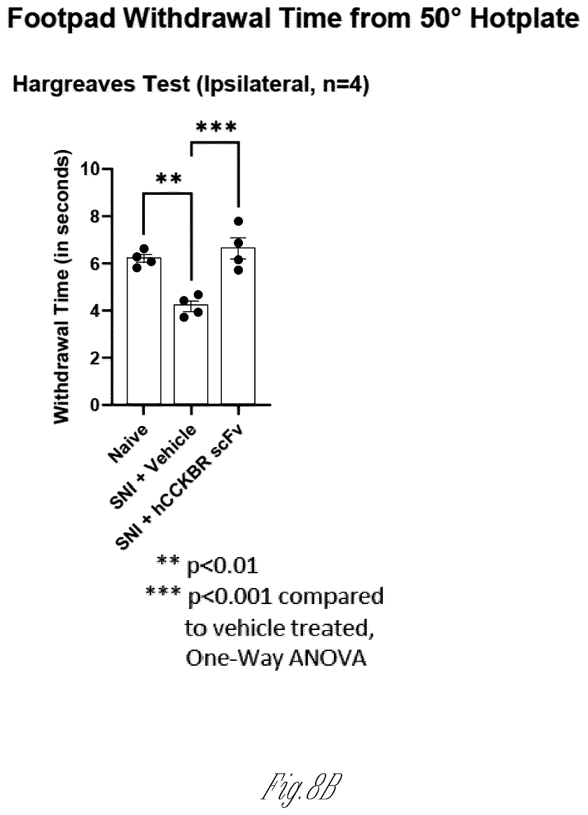

Mice with FRICT-ION were given daily injections of CCKBR inhibitor, LY225910 (s.c., 10 mg/kg). On Days 1-8, mice were tested prior to dosing. Efficacy tests on Days 6-8 progressed equivalently to a single dose of the CCKBR hscFv HC2-LC3 given on Day 1 (FIG. 6).
   Effect of CCKBR hscFv HC2-LC3 on CCKBR Immunostaining in Mouse DRG
Naïve mice have low levels of CCKBR protein immunostaining. FRICT-ION significantly increases the CCKBR immunostaining (10 weeks post). Treatment of FRICT-ION mice with a single dose of CCKBR hscFv HC2-LC3 in week 3 restores the baseline content of CCKBR immunostaining (week 10 post) (FIG. 7).
   Efficacy of Humanized CCKBR scFv HC2-LC3 in Other Models
The CCKBR scFv HC2-LC3 was efficacious in other pain and stress models.

### Sciatic Spared Nerve Injury (SNI) Chronic Pain Model

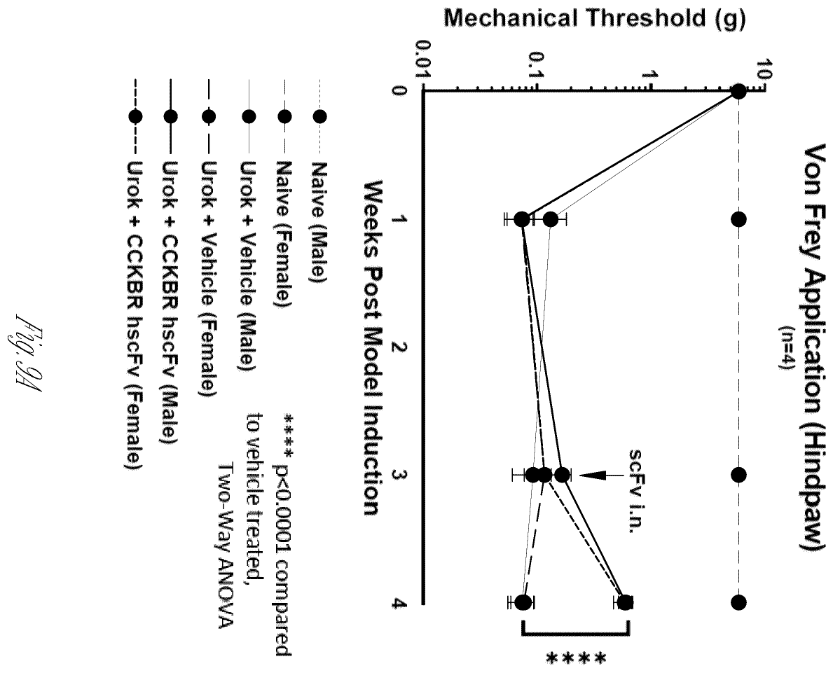

The sciatic spared nerve injury (SNI) rodent model is established in the literature and used by numerous labs in the field of pain research for study of mechanisms causal in chronic neuropathic pain (Decosterd & Woolf, 2000; Shields et al., 2003). The potential for effectiveness of the humanized CCKBR scFvs was tested in mice with SNI induced chronic neuropathic pain and followed through 10 weeks. The SNI model is induced in anesthetized mice by cutting the tibial and common peroneal branches of the sciatic nerve, while the sural nerve remains untouched. In sham operations, nerves are exposed but not ligated or cut and naïve mice remain untouched. All animals undergo baseline and weekly mechanical threshold testing. The SNI chronic pain model mice are given humanized scFv (4 mg/kg) or vehicle, intranasally (i.n) in week three post model induction.
Naïve mice received vehicle injection. The persisting effectiveness of the CCKBR scFvs for mechanical and heat hypersensitivity was followed through 10 weeks in the SNI chronic pain model (FIG. 8).

### Urokinase Back Pain Model

Low back pain is one of the most common causes of disability with 1 in 5 people suffering worldwide. In spite of this, few reliable animal models of back pain are popularly used in animal research in the pain field, especially in mice. The potential for effectiveness of the humanized CCKBR scFvs was tested in both male and female mice with urokinase induced chronic neuropathic pain. The rapid induction of a persisting back pain model in mice are provided here using injection of urokinase-type plasminogen activator (urokinase), a serine protease present in humans and other animals. The methodology for induction of persisting lower back pain in mice involves simple injection of urokinase along the ligamentous/intramuscular insertion region of the lumbar spine. The urokinase inflammatory agent is a serine protease that activates plasminogen to plasmin. Typically, the model can be induced within 10 minutes and hypersensitivity persists for at least 8-10 weeks. Hypersensitivity, gait disturbance and other standard, anxiety-and depression-like measures are be tested in the persisting model. These features are suitable for testing potential therapeutics aimed at reduction of back pain and its ancillary characteristics.

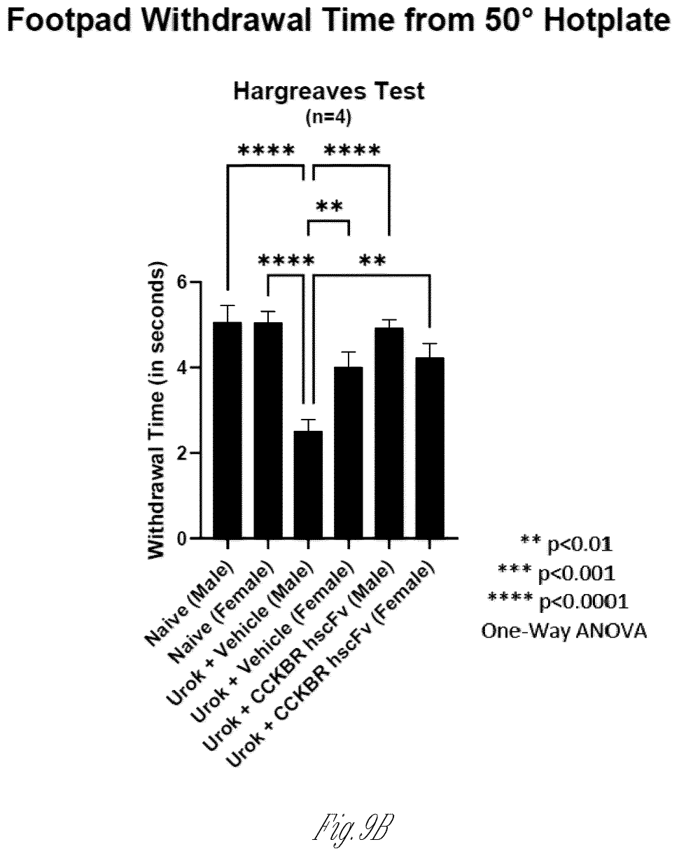

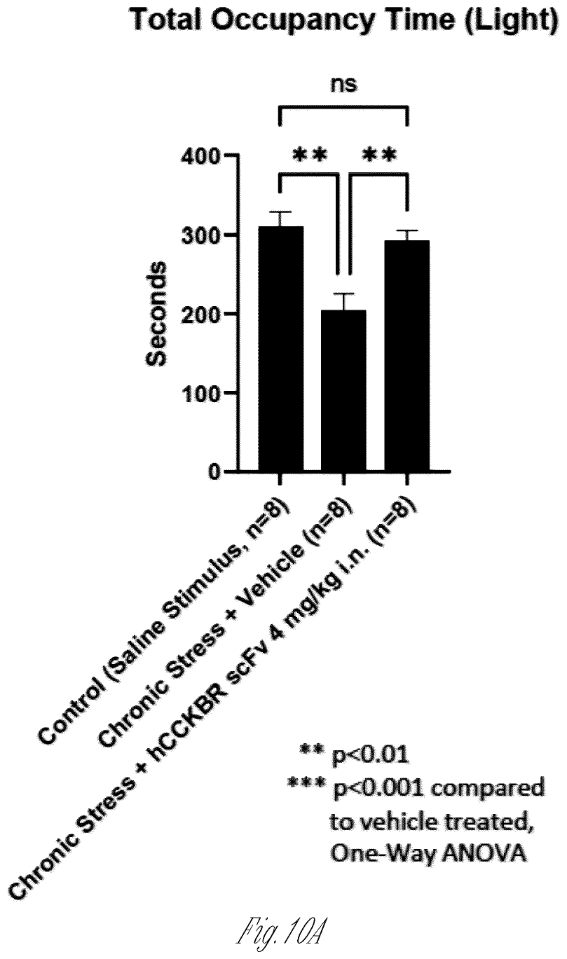

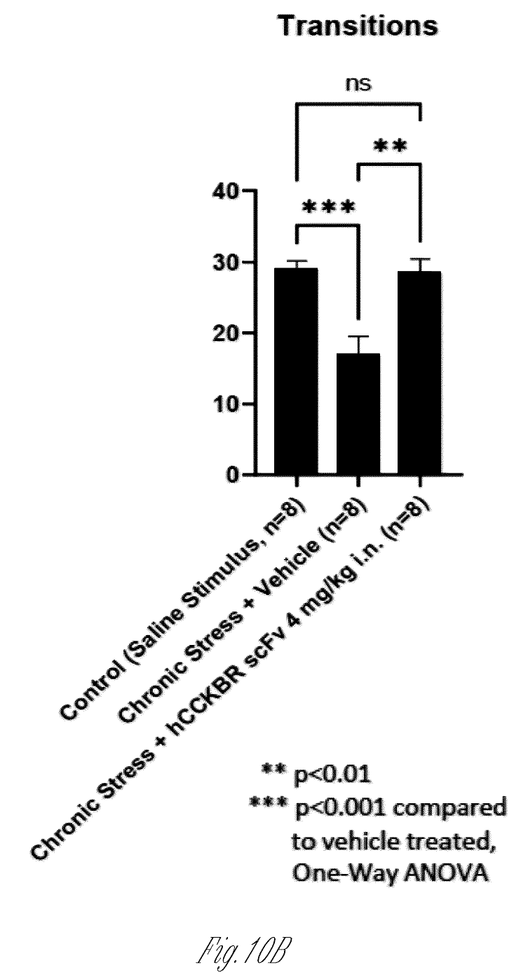

In mice, efficacy of the humanized CCKBR scFv HC2-LC3 was assessed in both male and female mice with urokinase induced back pain. Male and female mice were effectively relieved of their mechanical and heat hypersensitivity pain, particularly in the male mice with the back pain model (FIG. 9).

### Predator Stress Induced Anxiety

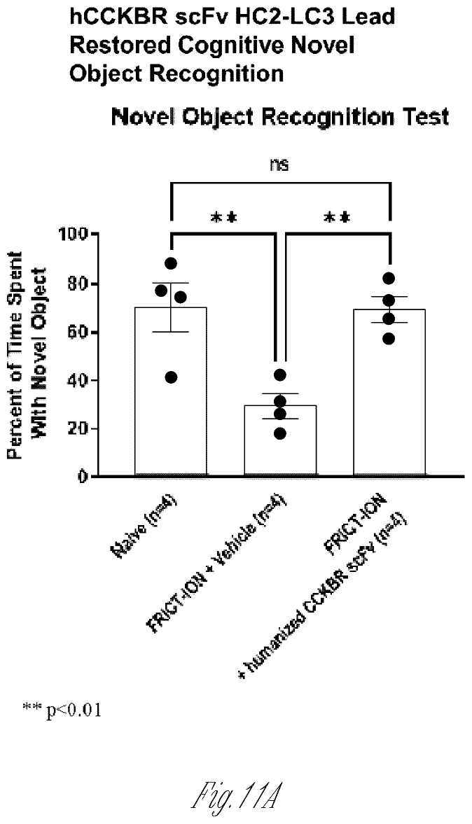

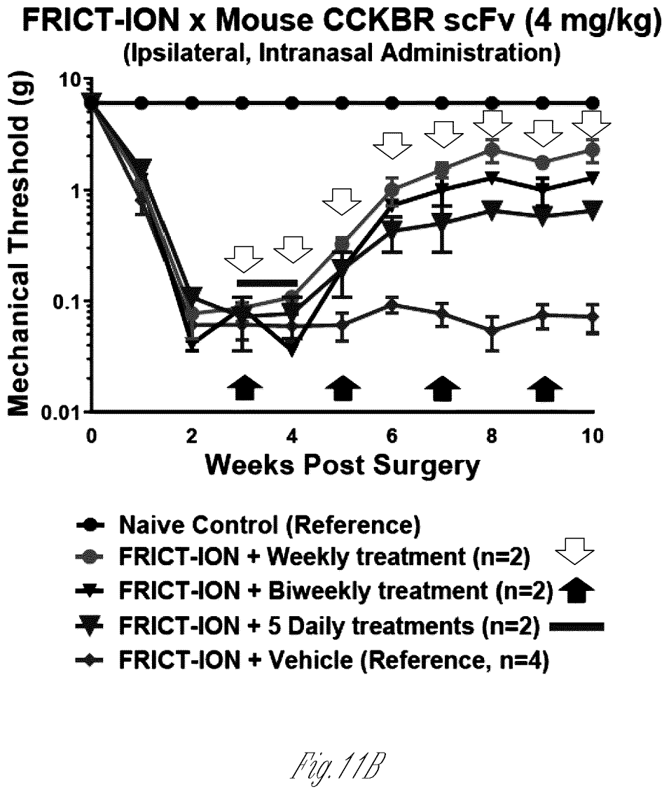

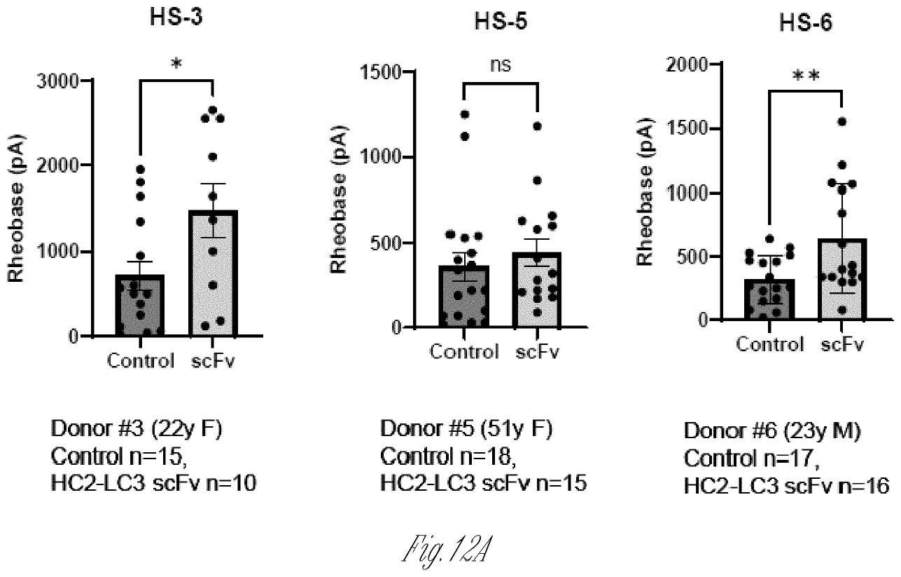

Mice were treated with the humanized CCKBR scFv HC2-LC3 injection was given to mice (control, sham, nerve injured). CCKBR scFv antibodies prevented anxiety-and depression-like behavior development in mice with nerve injury (FIG. 11). Cognitive assessment test with novel object recognition tests for the predator stress study indicated the scFv HC2-LC3 also prevented the cognitive disruption (FIG. 11).

### Experimental Behavioral Read-Outs

CCKBR scFvs were selected with the best binding affinity and tested for efficacy to reduce pain related measures using the following test read-outs.

Reflexive Mechanical Response Threshold Measurement Using von Frey Filaments. Hypersensitivity persists indefinitely in the FRICT-ION model, thus the model is suitable for assessing pain-like responses equivalent to the timeframe of chronic clinical pain (Montera & Westlund, 2020). Mechanical hypersensitivity was tested on the whiskerpad, the innervation territory of the infraorbital nerve, with von Frey filament stimulation at baseline and weekly thereafter as we have reported previously (Ma et al., 2012; Lyons et al., 2015, 2018; Montera & Westlund, 2020; Vigil et al., 2020). Reflexive responses to mechanical stimuli applied with graded von Frey filaments was tested weekly. A single trial consisted of 5 applications of several selected mid-range von Frey filaments applied once every 3 to 4 seconds. If no positive response is evoked, the next stronger filament is applied (Chaplan et al., 1994). Responses to decreased gram force filaments indicated increased hypersensitivity ((FIGS. 2, 5-6, 8-9, 11B)). A similar procedure is applied to the footpad if the back, sciatic nerve or muscle in the leg is injured.

Cognitive Dependent Anxiety-and Depression-Like Behaviors. The emotion based symptoms that develop in mice with persisting chronic pain did not develop in mice treated with humanized CCKBR scFv HC2-LC3 scFv. Tests of anxiety-and depression-like behaviors are tested in week 6-8 after induction of chronic pain model induction in treatment studies. Behaviors were video recorded for offline analysis.

Light/dark place preference test. Collected variables in this two chamber test were (1) time spent in each chamber, (2) number of transitions between chambers, (3) number of rearing events, and 4) entry latency into the light chamber (File et al., 2005; Wiley et al., 2007; Yalcin et al., 2014; Lyons et al., 2018).

Sucrose splash test. Depression-like behavior was tested with the sucrose splash test where decreased grooming behavior was defined as a measure of depression-like behavior (David et al., 2009; Yalcin et al., 2011). Depression-like behavior is tested by spritzing 10% sucrose solution (˜250 μl) on the mouse rump during 10 min. Mice with pain models do not show preference for the sweet treat. Naïve mice groom to retrieve the treat. The following may be measured: Number of Times Groomed, Total Groom Time or First Groom Latency.

Cognitive behavior test. Testing twice 2 days apart using different sets of Lego blocks provides an indication of the ability of mice to remember and explore new objects if not in pain or not explore novel objects if pain is present.

### Human Donor Dorsal Root Ganglia (DRG)

Human DRG were obtained from recently deceased organ donors after obtaining consent from next of kin. All procedures were approved by the Institutional Review Board (IRB) of the University of New Mexico Health Sciences Center according to study number 21-412 (PI: Alles, Sascha R). Human DRG were cultured for 2-10 days as previously described (Valtcheva et al., 2016). Whole-cell current clamp electrophysiological recordings were performed as previously described (Goins et al., 2022). Briefly, neurons were identified by infrared differential interference contrast (IR-DIC) connected to an IR2000 CCD camera (DageMTI, Michigan City, Indiana). Current-clamp recordings were performed using a Molecular Devices Multiclamp 700B (Scientifica, UK). Signals are filtered at 5 kHz, acquired at 50 kHz using a Molecular Devices 1550B converter (Scientifica, UK), and recorded using Clampex 11 software (Molecular Devices, Scientifica, UK). Electrodes were pulled with a Zeitz puller (Werner Zeitz, Martinsreid, Germany) from borosilicate thick glass (GC150F, Sutter Instruments). The resistance of the electrodes, following fire polishing of the tip, range between 5 and 8 MΩ. Bridge balance is applied to all recordings. Intracellular solution contains (in mM) 125 K-gluconate, 6 KCI, 10 HEPES, 0.1 EGTA, 2 Mg-ATP, pH 7.3 with KOH, and osmolarity of 290-310 mOsm. Artificial cerebrospinal fluid (aCSF) contains (in mM) 113 NaCl, 3 KCl, 25 NaHCO3, 1 NaH2PO4, 2 CaCl2, 2MgCl2, and 11 D-glucose.

### Direct Effects on Human Neurons

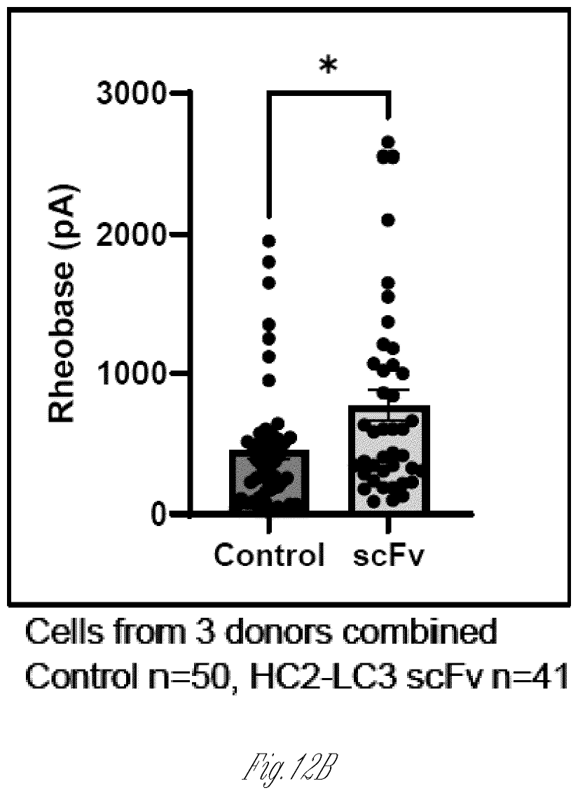

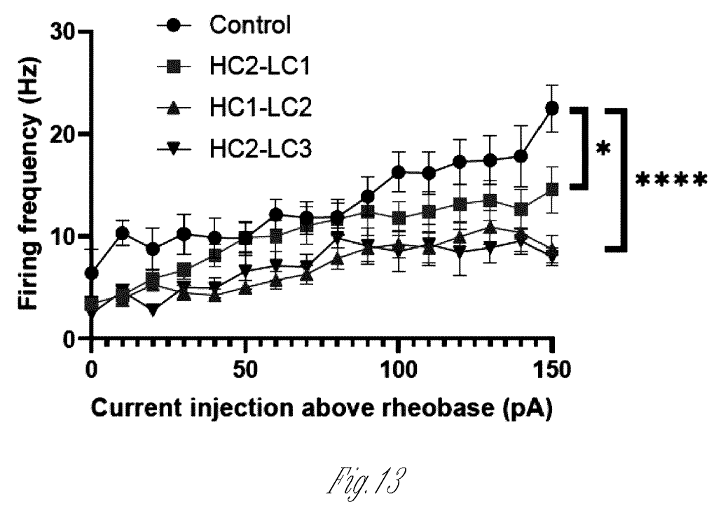

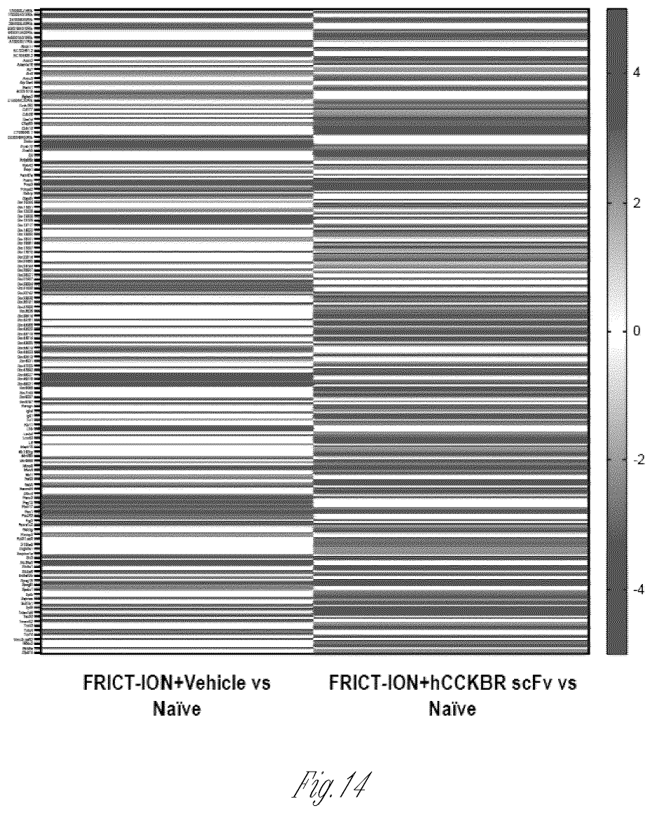

The methods for extraction and culture of human DRG neurons for whole-cell patch electrophysiology are shown in FIG. 13, along with neuronal firing characteristics. Data are shown in FIG. 14-15. Humanized CCKBR scFv (HC2-LC3) significantly reduces excitability (p<0.05, Mann-Whitney test) by increasing the rheobase (current required to elicit firing) of human dorsal root ganglia (hDRG) neurons as measured using whole-cell patch camp electrophysiology (FIG. 15). Effects of other humanized CCKBR scFv variants on human DRG neurons are shown in FIG. 15. In addition, 3 humanized variants (HC2-LC1, HC1-LC2 and HC2-LC3) significantly reduced the firing frequency of mouse DRG neurons (p<0.0001 ANOVA). This indicates reduced excitability of neurons treated with ₕᵤₘₐₙCCKBR scFv, corresponding to the reduction of pain measures in the mouse models.

The data thus demonstrate the effectiveness of the humanized CCKBR scFv in reducing excitability (increasing rheobase or reducing firing frequency) on human and mouse primary dorsal root ganglia (DRG) neurons. This demonstrates direct translational potential of the HC2-LC3 hscFv lead for effectiveness in patients with chronic pain in which the excitability of DRG neurons is increased.

### EXAMPLE 3

In one embodiment, an expression cassette is provided comprising nucleic acid sequences encoding an anti-CCKBR antibody or antigen binding fragment thereof, or a polypeptide, that inhibits human CCKBR activity, which sequence encodes at least one of SEQ ID NO:31, SEQ ID NO:32, SEQ ID NO:33, SEQ ID NO: 34, GAS, or SEQ ID NO:36, or a polypeptide with at least, 80% 85%, 90%, 92%, 94%, 95%, 96%, 97%, 98%, or 99% amino acid sequence identity thereto.

In one embodiment, an expression cassette is provided comprising nucleic acid sequences encoding an anti-CCKBR antibody or antigen binding fragment thereof, or a polypeptide, that inhibits human CCKBR activity, which sequence encodes a variable region having at least one of SEQ ID NO:1-LINKER-SEQ ID NO: 2, SEQ ID NO:3-LINKER-SEQ ID NO:4, SEQ ID NO:5-LINKER-SEQ ID NO: 6, SEQ ID NO:7-LINKER-SEQ ID NO:8, or a polypeptide with at least 80%, 85%, 90%, 92%, 94%, 95%, 96%, 97%, 98%, or 99% amino acid sequence identity thereto.

In one embodiment, an expression cassette is provided comprising nucleic acid sequences encoding an anti-CCKBR antibody or antigen binding fragment thereof, or a polypeptide, that inhibits human CCKBR activity, which sequence encodes a plurality of CDRs having at least one of SEQ ID NO:41, SEQ ID NO: 42, SEQ ID NO:43, or SEQ ID NO:44, a polypeptide with at least 80%, 85%, 90%, 92%, 94%, 95%, 96%, 97%, 98%, or 99% amino acid sequence identity thereto.

In one embodiment, an anti-CCKBR antibody or antigen binding fragment thereof, or a polypeptide, is provided that inhibits human CCKBR activity, which antibody or fragment thereof, or polypeptide, has at least one of SEQ ID NO: 31, SEQ ID NO:32, SEQ ID NO:33, SEQ ID NO:34, GAS, or SEQ ID NO: 36, or at least, 80% 85%, 90%, 92%, 94%, 95%, 96%, 97%, 98%, or 99% amino acid sequence identity thereto.

In one embodiment, an anti-CCKBR antibody or antigen binding fragment thereof, or a polypeptide, that inhibits human CCKBR activity, is provided, which has a variable region having at least one of SEQ ID NO:1-LINKER-SEQ ID NO: 2, SEQ ID NO:3-LINKER-SEQ ID NO:4, SEQ ID NO:5-LINKER-SEQ ID NO: 6, SEQ ID NO:7-LINKER-SEQ ID NO:8, or at least 80%, 85%, 90%, 92%, 94%, 95%, 96%, 97%, 98%, or 99% amino acid sequence identity thereto.

In one embodiment, an anti-CCKBR antibody or antigen binding fragment thereof, or a polypeptide, that inhibits human CCKBR activity, is provided which has a plurality of CDRs having at least one of SEQ ID NO:41, SEQ ID NO: 42, SEQ ID NO:43, or SEQ ID NO:44, or at least 80%, 85%, 90%, 92%, 94%, 95%, 96%, 97%, 98%, or 99% amino acid sequence identity thereto.

Exemplary sequences include:

```
scFv77-2 hu HC1-LC2
  (SEQ ID NO: 135)
MAQVQLVQSGAEVKKPGASVKVSCKASGFNIKDYYIHWVRQAPGQGL
EWIGWIDPENGDTEYAPKFQGRATMTADTSISTAYMELSRLRSDDTAVY
YCNAGGRFAYWGQGTLVTVSSGGGGSGGGGSGGGGSDIQMTQSPSSLS
ASVGDRVTITCKSSQSLLNSGNQKNYLAWYQQKPGKAPKLLIYGASTRE
SGVPSRFSGSGSGTDFTLTISSLQPEDFATYYCQNDHSYPYTFGGGTKL
EIK
scFv77-2 hu HC2-LC1
  (SEQ ID NO: 136)
MAQVQLVQSGAEVKKPGASVKVSCKASGFNIKDYYIHWVRQAPGQGL
EWIGWIDPENGDTEYAPKFQGRATMTADTSISTAYMELSRLRSDDTAVY
YCNAGGRFAYWGQGTLVTVSSGGGGSGGGGSGGGGSDIQMTQSPSSLS
ASVGDRVTITCKSSQSLLNSGNQKNYLAWYQQKPGKAPKLLIYGASTRE
SGVPSRFSGSGSGTDFTLTISSLQPEDFATYYCQNDHSYPYTFGGGTKL
EIK
scFv77-2 hu HC2-LC2
  (SEQ ID NO: 137)
MAQVQLVQSGAEVKKPGASVKVSCKASGFNIKDYYIHWVRQAPGQGL
EWIGWIDPENGDTEYAPKFQGRATMTADTSISTAYMELSRLRSDDTAVY
YCNAGGRFAYWGQGTLVTVSSGGGGSGGGGSGGGGSDIQMTQSPSSLS
ASVGDRVTITCKSSQSLLNSGNQKNYLAWYQQKPGKAPKLLIYGASTRE
SGVPSRFSGSGSGTDFTLTISSLQPEDFATYYCQNDHSYPYTFGGGTKL
EIK
scFv77-2 hu HC2-LC3
  (SEQ ID NO: 138)
MAQVQLVQSGAEVKKPGASVKVSCKASGFNIKDYYIHWVRQAPGQGL
EWIGWIDPENGDTEYAPKFQGRATMTADTSISTAYMELSRLRSDDTAVY
YCNAGGRFAYWGQGTLVTVSSGGGGSGGGGSGGGGSDIQMTQSPSTLS
ASVGDRVTITCKSSQSLLNSGNQKNYLAWYQQKPGKAPKLLIYGASTRE
SGVPSRFSGSGSGTEFTLTISSLQPDDFATYYCQNDHSYPYTFGGGTKV
EIK
scFv77-2 hu HC3-LC2
  (SEQ ID NO: 139)
MAQVQLVQSGAEVKKPGASVKVSCKASGFNIKDYYIHWVRQAPGQGL
EWIGWIDPENGDTEYAPKFQGRATMTADTSISTAYMELSRLRSDDTAVY
YCNAGGRFAYWGQGTLVTVSSGGGGSGGGGSGGGGSDIQMTQSPSSLS
ASVGDRVTITCKSSQSLLNSGNQKNYLAWYQQKPGKAPKLLIYGASTRE
SGVPSRFSGSGSGTDFTLTISSLQPEDFATYYCQNDHSYPYTFGGGTKL
EIK
```

### EXAMPLE 4

The disclosed humanized scFv have a binding affinity better than the murine parent scFv. The humanized scFv recognize a peptide fragment of human CCKBR. The scFv ware generated with cell-free ribosome display technology and recombinant antibody selection applied. In vivo validation in animal pain models with surgical induction of trigeminal nerve injury indicate that the humanized scFv is highly effective for reversing pain related behaviors to baseline within 2-3 weeks after a single administration (4 mg/kg, intraperitoneal). Many of the humanized scFvs have nanomolar binding affinity. The humanized scFv restore behavioral, physiological, and affective responses in two neuropathic pain models (sciatic and trigeminal nerve injury) that mimic human neuropathic pain conditions. Subsequent studies determine it use for reduction of leg muscle and back inflammatory pain.

Exemplary parental variable region sequences (murine) that bind CCKBR are (CDRs are highlighted and underlined):

```
mscFv77-2 Parental
Heavy chain:
  (SEQ ID NO: 10)
                                                    CDR1 CDR2
EVQLQQAGAELVRSGASVKLSCTASGFNIKDYYIHWVKQRPEQGLEWIGWIDPENGDTEYAPKF
QGKATMTADTSSNTAYLQLSSLTSEDTAVYYCNAGGRFAYWGQGTLVTVSA
                                      CDR3
Light chain:
  (SEQ ID NO: 11)
                                                        CDR1 CDR2
DIVMTQSPSSLSVSAGEKVTMSCKSSQSLLNSGNQKNYLAWYQQKPGQPPKLLIYGASTRESGVP
DRFTGSGSGTDFTLTISSVQAEDLAVYYCQNDHSYPYTFGGGTKLEIK.
                                    CDR3
```

Exemplary humanized variable region sequences that bind CCKBR are (CDRs are highlighted and underlined):

```
scFv77-2 hu HC1-linker-LC2
Heavy chain:
  (SEQ ID NO: 1)
QVQLVQSGAEVKKPGASVKVSCKASGFNIKDYYIHWVRQAPGQGLEWIGWID
PENGDTEYAPKFQGRATMTADTSISTAYMELSRLRSDDTAVYYCNAGGRFAY
WGQGTLVTVSS
Light chain:
  (SEQ ID NOI: 2)
DIQMTQSPSSLSASVGDRVTITCKSSQSLLNSGNQKNYLAWYQQKPGKAPKL
LIYGASTRESGVPSRFSGSGSGTDFTLTISSLQPEDFATYYCQNDHSYPYTFG
GGTKLEIK
scFv77-2 hu HC2-linker-LC1
Heavy chain:
  (SEQ ID NO: 3)
QVQLVQSGAEVKKPGASVKVSCKASGFNIKDYYIHWVRQATGQGLEWMGWI
DPENGDTEYAQKFQGRVTMTRDTSINTAYMELSSLRSEDTAVYYCNAGGRFA
YWGQGTLVTVSS
Light chain:
  (SEQ ID NO: 4)
DIQMTQSPSSLSASVGDRVTITCRASQSLLNSGNQKNYLAWYQQKPGKVPKL
LIYGASTRESGVPSRFSYSGSGTDFTLTISSLQPEDVWTYYCQNDHSYPYTFG
QGTKLEIK
scFv77-2 hu HC2-linker-LC3
Heavy chain:
  (SEQ ID NO: 5)
QVQLVQSGAEVKKPGASVKVSCKASGFNIKDYYIHWVRQATGQGLEWMGWI
DPENGDTEYAQKFQGRVTMTRDTSINTAYMELSSLRSEDTAVYYCNAGGRFA
YWGQGTLVTVSS
Light chain:
  (SEQ ID NO: 6)
DIVMTQSPDSLAVSLGERATINCKSSQSLLNSGNQKNYLAWYQQKPGQPWKL
LIYGASTRESGVPDRFSGSGSGTDFTLTISSLQAEDVYVYYCQNDHSYPYTFF
QGTKVEIK
scFv77-2 hu HC3-linker-LC3
Heavy chain:
  (SEQ ID NO: 7)
QVQLVQSGAEVKKPGASVKVSCKASGFNIKDYYIHWVRQAPGQGLEWIGWID
PENGDTEYAPKFQGRATMTADTSISTAYMELSRLRSDDTAVYYCNAGGRFAY
WGQGTLVTVSS
Light chain:
  (SEQ ID NO: 8)
DIQMTQSPSTLSASVGDRVTITCKSSQSLLNSGNQKNYLAWYQQKPGKAPKL
LIYGASTRESGVPSRFSGSGSGTEFTLTISSLQPDDFATYYCQNDHSYPYTFG
GGTKVEIK
```

Exemplary alignments of the heavy chain variable and light chain including variable humanized sequences are as follows:

```
Heavy
QVQLVQSGAEVKKPGASVKVSCKASGFNIKDYYIHWVRQAPGQGLEWIGWIDPE
QVQLVQSGAEVKKPGASVKVSCKASGFNIKDYYIHWVRQATGQGLEWMGWIDPE
QVQLVQSGAEVKKPGASVKVSCKASGFNIKDYYIHWVRQATGQGLEWMGWIDPE
QVQLVQSGAEVKKPGASVKVSCKASGFNIKDYYIHWVRQAPGQGLEWIGWIDPE
NGDTEYAPKFQGRATMTADTSISTAYMELSRLRSDDTAVYYCNAGGRFAYW
GQ
NGDPEYAQKFQGRVTMTRDTSINTAYMELSSLRSEDTAVYYCNAGGRFAYW
GQ
NGDPEYAQKFQGRVTMTRDTSINTAYMELSSLRSEDTAVYYCNAGGRFAYW
GQ
NGDTEYAPKFQGRATMTADTSISTAYMELSRLRSDDTAVYYCNAGGRFAYW
GQ
(SEQ ID NO: 1)
GTLVTVSS
(SEQ ID NO: 3)
GTLVTVSS
(SEQ ID NO: 5)
GTLVTVSS
  (SEQ ID NO: 7)
GTLVTVSS
Light
DIQMTQSPSSLSASVGDRVTITCKSSQSLLNSGNQKNYLAWYQQKPGKAPKL
LIY
DIQMTQSPSSLSASVGDRVTITCRASQSLLNSGNQKNYLAWYQQKPGKVPKL
LIY
DIVMTQSPDSLAVSLGERATINCKSSQSLLNSGNQKNYLAWYQQKPGQPWKL
LIY
DIQMTQSPSTLSASVGDRVTITCKSSQSLLNSGNQKNYLAWYQQKPGKAPKL
LIY
GASTRESGVPSRFSGSGSGTDFTLTISSLQPEDFATYYCQNDHSYPYTFGGG
TK
GASTRESGVPSRFSYSGSGTDFTLTISSLQPEDVWTYYCQNDHSYPYTFGQG
TK
GASTRESGVPDRFSGSGSGTDFTLTISSLQAEDVYVYYCQNDHSYPYTFFQG
TK
GASTRESGVPSRFSGSGSGTEFTLTISSLQPDDFATYYCQNDHSYPYTFGGG
TK
(SEQ ID NO: 2)
LEIK
  (SEQ ID NO: 4)
LEIK
  (SEQ ID NO: 6)
VEIK
(SEQ ID NO: 8)
VEIK
```

An exemplary linker sequence comprises SSGGGGSGGGGSGGGG (SEQ ID NO:9) or GGGGSGGGGGGGGSS (SEQ ID NO:22).

Exemplary scFv having heavy or light chain variable regions wherein the CDRs (DRs) disclosed above may include, for example, a polypeptide having:

(SEQ ID NO: 41)
QVQLVQSGAEVKKPGASVKVSCKAS
[CDRHC1]IHWVRQAPGQGLEWIG
[CDRHC2]EYAPKFQGRATMTADTSISTAYMELSRLRSDDTAVYYC
[CDRHC3]WGQGTLVTVSSLINKERDIQMTQSPSSLSASVGDRVTI
TCKSS[CDRLC1]LAWYQQKPGKAPKLLI[CDRLC2]TRESGVPSR
FSGSGSGTDFTLTISSLQPEDFATYYC[CDRL3]FGGGTKLEIK,

or a polypeptide with at least 80%, 85%, 90%, 92%, 94%, 95%, 96%, 97%, 98%, or 99% amino acid sequence identity thereto in the non-CDR region(s), e.g., 1, 2, 3 4 or 5 substitutions.

(SEQ ID NO: 42)
QVQLVQSGAEVKKPGASVKVSCKAS
[CDRHC1]IHWVRQATGQGLEWMGW[CDRHC2]
EYAQKFQGRVTMTRDTSINTAYMELSSLRSEDTAVYYC[CDRHC3]
WGQGTLVTVSSLINKER DIQMTQSPSSLSASVGDRVTITCRAS
[CDRLC1]LAWYQQKPGKVPKLLIY[CDRHLC2]
TRESGVPSRFSYSGSGTDFTLTISSLQPEDVWTYYC[CDRLC3]
FGQGTKLEIK,

or a polypeptide with at least 80%, 85%, 90%, 92%, 94%, 95%, 96%, 97%, 98%, or 99% amino acid sequence identity thereto in the non-CDR region(s), e.g., 1, 2, 3 4 or 5 substitutions.

(SEQ ID NO: 43)
QVQLVQSGAEVKKPGASVKVSCKAS
[CDRHC1]IHWVRQATGQGLEWMGW[CDRHC2]
EYAQKFQGRVTMTRDTSINTAYMELSSLRSEDTAVYYCCDRHC3]
WGQGTLVTVSSLINKERDIVMTQSPDSLAVSLGERATINCKS[CDRLC1]
LAWYQQKPGQPWKLLIY[CDRLC2]
TRESGVPDRFSGSGSGTDFTLTISSLQAEDVYVYY[CDRLC3]
FFQGTKVEIK,

or a polypeptide with at least 80%, 85%, 90%, 92%, 94%, 95%, 96%, 97%, 98%, or 99% amino acid sequence identity thereto in the non-CDR region(s), e.g., 1, 2, 3 4 or 5 substitutions.

(SEQ ID NO: 44)
QVQLVQSGAEVKKPGASVKVSCKAS
[CDRHC1]IHWVRQAPGQGLEWIGW
[CDRHC2]YAPKFQGRATMTADTSISTAYMELSRLRSDDTAVY
YCCDRHC3]
WGQGTLVTVSSLINKERDIQMTQSPSTLSASVGDRVTITCKSS
[CDRLC1]
LAWYQQKPGKAPKLLIY
[CDRLC2]TRESGVPSRFSGSGSGTEFTLTISSLQPDDFATYYC
[CDRLC3]FGGGTKVEIK,

or a polypeptide with at least 80%, 85%, 90%, 92%, 94%, 95%, 96%, 97%, 98%, or 99% amino acid sequence identity thereto in the non-CDR region(s), e.g., 1, 2, 3 4 or 5 substitutions.

(SEQ ID NO: 96)
MAQVQLVQSGAEVKKPGASVKVSCKAS[CDRHC1]IHWVRQAPGQGLEW
IGW[CDRHC2]EYAPKFQGRATMTADTSISTAYMELSRLRSDDTAVYYC
[CDRHC3]WGQGTLVTVSSLINKERDIQMTQSPSSLSASVGDRVTITCK
SS
[CDRLC1]LAWYQQKPGKAPKLLIY[CDRLC2]TRESGVPSRFSGSGSGT
DFTLTISSLQPEDFATYY[CDRLC3]FGGGTKLEIKENLYFQGAAALE,

or a polypeptide with at least 80%, 85%, 90%, 92%, 94%, 95%, 96%, 97%, 98%, or 99% amino acid sequence identity thereto in the non-CDR region(s), e.g., 1, 2, 3 4 or 5 substitutions.

(SEQ ID NO: 97)
MAQVQLVQSGAEVKKPGASVKVSC[CDRHC1]IHWVRQAPGQGLEWIGW
[CDRHC2]YAPKFQGRATMTADTSISTAYMELSRLRSDDTAVYYC
[CDRHC3]NAGGRFAYWGQGTLVTVSSLINKERDIQMTQSPSSLSASVG
DRVTITCKSS[CDRLC1]LAWYQQKPGKAPKLLIY[CDRLC2]TRESGV
PSRFSGSGSGTDFTLTISSLQPEDFATYY[CDRLC3]FGGGTKLEIKEN
LYFQGAAALE,

or a polypeptide with at least 80%, 85%, 90%, 92%, 94%, 95%, 96%, 97%, 98%, or 99% amino acid sequence identity thereto in the non-CDR region(s), e.g., 1, 2, 3 4 or 5 substitutions.

Exemplary scFv having heavy or light chain variable regions, having the CDRs (DRs) disclosed above or other CDRs, may include, for example, a polypeptide having one of

```
(SEQ ID NO: 51)
QVQLVQSGAEVKKPGASVKVSCKAS,
(SEQ ID NO: 52)
IHWVRQAPGQGLEWIG,
(SEQ ID NO: 53)
EYAPKFQGRATMTADTSISTAYMELSRLRSDDTAVYYC,
(SEQ ID NO: 54)
WGQGTLVTVSS,
(SEQ ID NO: 55)
DIQMTQSPSSLSASVGDRVTITCKSS,
(SEQ ID NO: 56)
LAWYQQKPGKAPKLLI,
(SEQ ID NO: 57)
TRESGVPSRFSGSGSGTDFTLTISSLQPEDFATYYC,
(SEQ ID NO: 58)
FGGGTKLEIK,
(SEQ ID NO: 59)
QVQLVQSGAEVKKPGASVKVSCKAS,
(SEQ ID NO: 60)
IHWVRQATGQGLEWMGW,
(SEQ ID NO: 61)
EYAQKFQGRVTMTRDTSINTAYMELSSLRSEDTAVYYC,
(SEQ ID NO: 62)
WGQGTLVTVSS,
  (SEQ ID NO: 63)
DIQMTQSPSSLSASVGDRVTITCRAS,
(SEQ ID NO: 64)
LAWYQQKPGKVPKLLIY,
(SEQ ID NO: 65)
TRESGVPSRFSYSGSGTDFTLTISSLQPEDVWTYYC,
(SEQ ID NO: 66)
FGQGTKLEIK,
(SEQ ID NO: 67)
QVQLVQSGAEVKKPGASVKVSCKAS,
  (SEQ ID NO: 68)
IHWVRQATGQGLEWMGW,
(SEQ ID NO: 69)
EYAQKFQGRVTMTRDTSINTAYMELSSLRSEDTAVYYC,
  (SEQ ID NO: 70)
WGQGTLVTVSS,
  (SEQ ID NO: 71)
DIVMTQSPDSLAVSLGERATINCKS,
  (SEQ ID NO: 72)
LAWYQQKPGQPWKLLIY,
  (SEQ ID NO: 73)
TRESGVPDRFSGSGSGTDFTLTISSLQAEDVYVYY,
  (SEQ ID NO: 74)
FFQGTKVEIK,
  (SEQ ID NO: 75)
QVQLVQSGAEVKKPGASVKVSCKAS,
  (SEQ ID NO: 76)
IHWVRQAPGQGLEWIGW,
  (SEQ ID NO: 77)
YAPKFQGRATMTADTSISTAYMELSRLRSDDTAVYYC,
  (SEQ ID NO: 78)
WGQGTLVTVSS,
  (SEQ ID NO: 79)
DIQMTQSPSTLSASVGDRVTITCKSS,
  (SEQ ID NO: 80)
LAWYQQKPGKAPKLLIY,
  (SEQ ID NO: 81)
TRESGVPSRFSGSGSGTEFTLTISSLQPDDFATYYC,
  (SEQ ID NO: 82)
FGGGTKVEIK,
  (SEQ ID NO: 100)
DIQMTQSPSSLSASVGDRVTITCKSS,
  (SEQ ID NO: 101)
LAWYQQKPGKAPKLLIY,
  (SEQ ID NO: 102)
TRESGVPSRFSGSGSGTDFTLTISSLQPEDFATYYC,
  (SEQ ID NO: 103)
FGGGTKLEIKENLYFQGAAALE,
  (SEQ ID NO: 104)
MAQVQLVQSGAEVKKPGASVKVSCKAS,
  (SEQ ID NO: 105)
IHWVRQAPGQGLEWIGW,
(SEQ ID NO: 106)
EYAPKFQGRATMTADTSISTAYMELSRLRSDDTAVYYC,
  (SEQ ID NO: 107)
WGQGTLVTVSS,
  (SEQ ID NO: 108)
DIQMTQSPSTLSASVGDRVTITCKSS,
  (SEQ ID NO: 109)
LAWYQQKPGKAPKLLIY,
  (SEQ ID NO: 110)
TRESGVPSRFSGSGSGTEFTLTISSLQPDDFATYYC,
  (SEQ ID NO: 111)
FGGGTKVEIKENLYFQGAAALE,
```

or a polypeptide with at least 80%, 85%, 90%, 92%, 94%, 95%, 96%, 97%, 98%, or 99% amino acid sequence identity thereto, e.g., one having 1, 2, 3 4 or 5 substitutions.

### EXAMPLE 5

In one embodiment, a vector is provided comprising nucleic acid sequences encoding an anti-CCKBR antibody or antigen binding fragment thereof, or a polypeptide, that inhibits human CCKBR activity, which sequence encodes at least one of SEQ ID NO:31, SEQ ID NO:32, SEQ ID NO:33, SEQ ID NO: 34, GAS, or SEQ ID NO:36, or a polypeptide with at least, 80% 85%, 90%, 92%, 94%, 95%, 96%, 97%, 98%, or 99% amino acid sequence identity thereto.

In one embodiment, a vector is provided comprising nucleic acid sequences encoding an anti-CCKBR antibody or antigen binding fragment thereof, or a polypeptide, that inhibits human CCKBR activity, which sequence encodes a variable region having at least one of SEQ ID NO:1-LINKER-SEQ ID NO: 2, SEQ ID NO:3-LINKER-SEQ ID NO:4, SEQ ID NO:5-LINKER-SEQ ID NO: 6, SEQ ID NO:7-LINKER-SEQ ID NO:8, or a polypeptide with at least 80%, 85%, 90%, 92%, 94%, 95%, 96%, 97%, 98%, or 99% amino acid sequence identity thereto.

In one embodiment, a vector is provided comprising nucleic acid sequences encoding an anti-CCKBR antibody or antigen binding fragment thereof, or a polypeptide, that inhibits human CCKBR activity, which sequence encodes a plurality of CDRs having at least one of SEQ ID NO:41, SEQ ID NO: 42, SEQ ID NO:43, or SEQ ID NO:44, a polypeptide with at least 80%, 85%, 90%, 92%, 94%, 95%, 96%, 97%, 98%, or 99% amino acid sequence identity thereto.

In one embodiment, an anti-CCKBR antibody or antigen binding fragment thereof, or a polypeptide, is provided that inhibits human CCKBR activity, which antibody or fragment thereof, or polypeptide, has at least one of SEQ ID NO: 31, SEQ ID NO:32, SEQ ID NO:33, SEQ ID NO:34, GAS, or SEQ ID NO: 36, or at least, 80% 85%, 90%, 92%, 94%, 95%, 96%, 97%, 98%, or 99% amino acid sequence identity thereto.

In one embodiment, an anti-CCKBR antibody or antigen binding fragment thereof, or a polypeptide, that inhibits human CCKBR activity, is provided, which has a variable region having at least one of SEQ ID NO:1-LINKER-SEQ ID NO: 2, SEQ ID NO:3-LINKER-SEQ ID NO:4, SEQ ID NO:5-LINKER-SEQ ID NO: 6, SEQ ID NO:7-LINKER-SEQ ID NO:8, or at least 80%, 85%, 90%, 92%, 94%, 95%, 96%, 97%, 98%, or 99% amino acid sequence identity thereto.

In one embodiment, an anti-CCKBR antibody or antigen binding fragment thereof, or a polypeptide, that inhibits human CCKBR activity, is provided which has a plurality of CDRs having at least one of SEQ ID NO:41, SEQ ID NO: 42, SEQ ID NO:43, or SEQ ID NO:44, or at least 80%, 85%, 90%, 92%, 94%, 95%, 96%, 97%, 98%, or 99% amino acid sequence identity thereto.

### EXAMPLE 6

Human dorsal root ganglia (DRG) were obtained from recently deceased organ donors after obtaining consent from next of kin. All procedures were approved by the Institutional Review Board (IRB) of the University of New Mexico Health Sciences Center according to study number 21-412 (PI: Alles, Sascha R). Human DRG were cultured for 2-10 days as previously described (Valtcheva et al., 2016). Whole-cell current clamp electrophysiological recordings were performed as previously described (Goins et al., 2022). Briefly, neurons were identified by infrared differential interference contrast (IR-DIC) connected to an IR2000 CCD camera (DageMTI, Michigan City, Indiana). Current-clamp recordings were performed using a Molecular Devices Multiclamp 700B (Scientifica, UK). Signals are filtered at 5 kHz, acquired at 50 kHz using a Molecular Devices 1550B converter (Scientifica, UK), and recorded using Clampex 11 software (Molecular Devices, Scientifica, UK). Electrodes were pulled with a Zeitz puller (Werner Zeitz, Martinsreid, Germany) from borosilicate thick glass (GC150F, Sutter Instruments). The resistance of the electrodes, following fire polishing of the tip, range between 5 and 8 MQ. Bridge balance is applied to all recordings. Intracellular solution contains (in mM) 125 K-gluconate, 6 KCl, 10 HEPES, 0.1 EGTA, 2 Mg-ATP, pH 7.3 with KOH, and osmolarity of 290-310 mOsm. Artificial cerebrospinal fluid (aCSF) contains (in mM) 113 NaCl, 3 KCl, 25 NaHCO3, 1 NaH2PO4, 2 CaCl2, 2 MgCl2, and 11 D-glucose.

### Direct Effects on Human Neurons

Humanized CCKBR scFv (HC2-LC3) reduces excitability of human dorsal root ganglia (hDRG) neurons tested with patch camp electrodes positioned for whole-cell patch clamp recordings. Multi-firing response measured using current clamp of a hDRG neuron to current injection were less numerous. Effect of hCCKBR scFv treatment on resting membrane potential

(RMP), show a hyperpolarizing effect compared to controls, and rheobase (current required to elicit firing), show an increase compared to controls. This indicates reduced excitability of neurons treated with ₕᵤₘₐₙCCKBR scFv, corresponding to the reduction of pain.

The data thus demonstrate the effectiveness of the humanized CCKBR scFv in reducing excitability (increasing rheobase or hyperpolarizing resting membrane potential) on human primary dorsal root ganglia (DRG) neurons. This demonstrates direct translational potential of the scFv for effectiveness in patients with chronic pain in which the excitability of DRG neurons is increased.

### REFERENCES

Anderson L C, Vakoula A, Veinote R. Inflammatory hypersensitivity in a rat model of trigeminal neuropathic pain. Arch Oral Biol. 2003 February;48 (2): 161-9. doi: 10.1016/s0003-9969 (02) 00203-0. PMID: 12642236.
Chaplan S R, Bach F W, Pogrel J W, Chung J M, Yaksh T L. Quantitative assessment of tactile allodynia in the rat paw. J Neurosci Methods. 1994 July; 53 (1): 55-63. doi: 10.1016/0165-0270 (94) 90144-9. PMID: 7990513.
David D J, Samuels B A, Rainer Q, et al. Neurogenesis-dependent and-independent effects of fluoxetine in an animal model of anxiety/depression. Neuron. 2009; 62 (4): 479-493. doi: 10.1016/j.neuron.2009.04.017
Decosterd & Woolf, 2000
Ding W, You Z, Shen S, Yang J, Lim G, Doheny J T, Chen L, Zhu S, Mao J. An Improved Rodent Model of Trigeminal Neuropathic Pain by Unilateral Chronic Constriction Injury of Distal Infraorbital Nerve. J Pain. 2017 August; 18(8): 899-907. doi: 10.1016/j.jpain.2017.02.427. Epub 2017 Feb. 24. PMID: 28238950; PMCID: PMC5537008.
Dutta S, Sengupta P. Men and mice: Relating their ages. Life Sci. 2016 May 1; 152: 244-8. doi: 10.1016/j.lfs.2015.10.025. PMID: 26596563.
File S E, Lippa A S, Beer B, Lippa M T (2005) Animal tests of anxiety. Curr Protoc Pharmacol. Chapter 5: Unit 5.38. doi:
10.1002/0471141755.ph0538s27. PMID: 22294128
Goins A E, Gomez K, Ran D, Afaghpour-Becklund M, Khanna R, Alles S R A.
   Neuronal allodynic mechanisms of Slc7a5 (LAT1) in the spared nerve injury rodent model of neuropathic pain. Pflugers Arch. 2022 April; 474(4): 397-403.doi: 10.1007/s00424-021-02653-9. Epub 2022 Jan. 20. PMID: 35048187;
   PMCID: PMC8930528.
Imamura Y, Kawamoto H, Nakanishi O. Characterization of heat-hyperalgesia in an experimental trigeminal neuropathy in rats. Exp Brain Res. 1997 August; 116(1): 97-103. doi: 10.1007/p100005748. PMID: 9305818.
Kozakov D, Brenke R, Comeau S R, Vajda S. PIPER: an FFT-based protein docking program with pairwise potentials. Proteins. 2006 Nov. 1; 65(2): 392-406. doi: 10.1002/prot.21117. PMID: 16933295.
Kunamneni, A., et al. (2019a) Generation and Selection of a Panel of Pan-Filovirus Single-Chain Antibodies using Cell-Free Ribosome Display. Am J Trop Med Hyg.
Kunamneni A., et al. (2019b) Generating highly potent and efficacious antibodies to the cholecystokinin B (CCK-B) receptor by ribosome display for the treatment of neuropathic pain. The FASEB J, 3, 1b31-1b31.
Kunamneni, A., et al. (2018) Ribosome display for the rapid generation of high-affinity Zika-neutralizing single-chain antibodies. PLOS One, 13, e0205743.
Lyons D N, Kniffin T C, Zhang L P, et al. Trigeminal Inflammatory Compression (TIC) injury induces chronic facial pain and susceptibility to anxiety-related behaviors. Neuroscience. 2015; 295: 126-138. doi: 10.1016/j.neuroscience.2015.03.051
Lyons D N, Zhang L, Pandya J D, et al. Combination Drug Therapy of Pioglitazone and D-cycloserine Attenuates Chronic Orofacial Neuropathic Pain and Anxiety by Improving Mitochondrial Function Following Trigeminal Nerve Injury. Clin J Pain. 2018; 34(2): 168-177. doi: 10.1097/AJP.0000000000000515
Ma F, Zhang L, Lyons D, Westlund K N. Orofacial neuropathic pain mouse model induced by Trigeminal Inflammatory Compression (TIC) of the infraorbital nerve. Mol Brain. 2012 ;5: 44. Published 2012 Dec. 28. doi: 10.1186/1756-6606-5-44
Montera M A, Westlund K N. Minimally Invasive Oral Surgery Induction of the FRICT-ION Chronic Neuropathic Pain Model. Bio Protoc. 2020 Apr. 20; 10(8): e3591. doi: 10.21769/BioProtoc.3591. PMID: 33659557; PMCID: PMC7842532.
Okubo M, Castro A, Guo W, Zou S, Ren K, Wei F, Keller A, Dubner R. Transition to persistent orofacial pain after nerve injury involves supraspinal serotonin mechanisms. J Neurosci. 2013 Mar. 20; 33(12): 5152-61. doi: 10.1523/JNEUROSCI.3390-12.2013. PMID: 23516281; PMCID: PMC3640487.
Rikard S M, Strahan A E, Schmit K M, Guy G P Jr. Chronic Pain Among Adults-United States, 2019-2021. MMWR Morb Mortal Wkly Rep. 2023 April 14; 72(15): 379-385. doi: 10.15585/mmwr.mm7215a1. PMID: 37053114; PMCID: PMC10121254.
Seltzer Z, Dubner R, Shir Y. A novel behavioral model of neuropathic pain disorders produced in rats by partial sciatic nerve injury. Pain. 1990; 43: 245-250.
Shields et al., 2003
Tall J M, Raja S N. Dietary constituents as novel therapies for pain. Clin J Pain. 2004 January-Feb; 20(1): 19-26. doi: 10.1097/00002508-200401000-00005. PMID: 14668652.
Tang, Y.; Cao, Y. Modeling Pharmacokinetics and Pharmacodynamics of Therapeutic Antibodies: Progress, Challenges, and Future Directions. Pharmaceutics 2021, 13, 422. https://doi.org/10.3390/pharmaceutics 13030422
Valtcheva M V, Copits B A, Davidson S, Sheahan T D, Pullen M Y, McCall J G, Dikranian K, Gereau R W 4th. Surgical extraction of human dorsal root ganglia from organ donors and preparation of primary sensory neuron cultures. Nat Protoc. 2016 October; 11(10): 1877-88. doi: 10.1038/nprot.2016.111. Epub 2016 Sep. 8. PMID: 27606776; PMCID: PMC5082842.
Vigil J M, Montera M A, Pentkowski N S, Diviant J P, Orozco J, Ortiz A L, Rael L J, Westlund K N. The Therapeutic Effectiveness of Full Spectrum Hemp Oil Using a Chronic Neuropathic Pain Model. Life (Basel). 2020 May 18; 10(5): 69. doi: 10.3390/life10050069. PMID: 32443500; PMCID: PMC7281216.
Vos B P, Strassman A M, Maciewicz R J. Behavioral evidence of trigeminal neuropathic pain following chronic constriction injury to the rat's infraorbital nerve. J Neurosci. 1994 May; 14(5 Pt 1): 2708-23. doi: 10.1523/JNEUROSCI.14-05-02708.1994. PMID: 8182437
Wallace A C, Laskowski R A, Thornton J M. LIGPLOT: a program to generate schematic diagrams of protein-ligand interactions. Protein Eng. 1995 February; 8(2): 127-34. doi: 10.1093/protein/8.2.127. PMID: 7630882.
Westlund K N, Montera M A, Goins A E, Alles S R A, Afaghpour-Becklund M, Bartel R, Durvasula R, Kunamneni A. Single-chain Fragment variable antibody targeting cholecystokinin-B receptor for pain reduction. Neurobiol Pain. 2021 Jul. 15; 10: 100067. doi: 10.1016/j.ynpai.2021.100067. PMID: 34458647; PMCID: PMC8378781.
Wiley R G, Kline R H 4th, Vierck C J Jr. Anti-nociceptive effects of selectively destroying substance P receptor-expressing dorsal horn neurons using [Sar9,Met(O2)11]-substance P-saporin: behavioral and anatomical analyses. Neuroscience. 2007 May 25; 146(3): 1333-45. doi: 10.1016/j.neuroscience.2007.01.066. Epub 2007 Apr. 5. PMID: 17418497.
Xu M, Aita M, Chavkin C. Partial infraorbital nerve ligation as a model of trigeminal nerve injury in the mouse: behavioral, neural, and glial reactions. J Pain. 2008 November; 9(11): 1036-48. doi: 10.1016/j.jpain.2008.06.006. PMID: 18708302; PMCID: PMC2632609.
Xu, D. & Zhang, Y, 2011. and fragment-guided molecular dynamics (FG-MD) simulation Biophys J 101 (10): 2525-2534.
Yalcin I, Bohren Y, Waltisperger E, Sage-Ciocca D, Yin J C, Freund-Mercier M J, Barrot M. A time-dependent history of mood disorders in a murine model of neuropathic pain. Biol Psychiatry. 2011 Nov. 15; 70(10): 946-53. doi: 10.1016/j.biopsych.2011.07.017. Epub 2011 Sep. 3. PMID: 21890110.
Yalcin I, Megat S, Barthas F, et al. The sciatic nerve cuffing model of neuropathic pain in mice. J Vis Exp. 2014; (89): 51608. Published 2014 Jul. 16. doi: 10.3791/51608
Yang J, Zhang W, He B, Walker S E, Zhang H, Govindarajoo B, Virtanen J, Xue Z, Shen H B, Zhang Y. Template-based protein structure prediction in CASP11 and retrospect of I-TASSER in the last decade. Proteins. 2016 September; 84 Suppl 1(Suppl 1): 233-46. doi: 10.1002/prot.24918. PMID: 26343917; PMCID: PMC4781680.
Zhang J, Liang Y, Zhang Y. Atomic-level protein structure refinement using fragment-guided molecular dynamics conformation sampling. Structure. 2011 Dec. 7 ;19(12): 1784-95. doi: 10.1016/j.str.2011.09.022. PMID: 22153501; PMCID: PMC3240822.Zhang Y. I-TASSER server for protein 3D structure prediction. BMC Bioinformatics. 2008; 9: 40. Published 2008 Jan. 23. doi: 10.1186/1471-2105-9-40.
Zhu K, Day T, Warshaviak D, Murrett C, Friesner R, Pearlman D. Antibody structure determination using a combination of homology modeling, energy-based refinement, and loop prediction. Proteins. 2014; 82(8): 1646-1655. doi: 10.1002/prot.24551
All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising a humanized anti-human CCKBR antibody, or an antigen binding fragment thereof, or a polypeptide, that inhibits human CCKBR activity, wherein the antibody, the antigen binding fragment thereof, or the polypeptide has:
i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO: 33); and/or
ii) a variable region comprising a first CDR comprising QSLLNSGNQKNY (SEQ ID NO: 34) operably linked to a second CDR comprising GAS operably linked to a third CDR comprising QNDHSYPYT (SEQ ID NO:36).

2. The composition of claim 2 wherein the antibody is a scFv.

3. The composition of claim 1 wherein the first, second, or third CDR, or any combination thereof, of variable region i) is/are flanked by one or more human Ig framework sequences.

4. The composition of claim 3 wherein the one or more human Ig framework sequences comprise QVQLVQSGAEVKKPGASVKVSCKAS (SEQ ID NO:51), IHWVRQAPGQGLEWIG (SEQ ID NO:52), EYAPKFQGRATMTADTSISTAYMELSRLRSDDTAVYYC (SEQ ID NO:53), WGQGTLVTVSS (SEQ ID NO:54), QVQLVQSGAEVKKPGASVKVSCKAS (SEQ ID NO: 59), IHWVRQATGQGLEWMGW (SEQ ID NO:60), EYAQKFQGRVTMTRDTSINTAYMELSSLRSEDTAVYYC (SEQ ID NO:61), WGQGTLVTVSS (SEQ ID NO:62), QVQLVQSGAEVKKPGASVKVSCKAS (SEQ ID NO: 67), IHWVRQATGQGLEWMGW (SEQ ID NO:68), EYAQKFQGRVTMTRDTSINTAYMELSSLRSEDTAVYYC (SEQ ID NO:69), WGQGTLVTVSS (SEQ ID NO:70), QVQLVQSGAEVKKPGASVKVSCKAS (SEQ ID NO: 75), IHWVRQAPGQGLEWIGW (SEQ ID NO:76), YAPKFQGRATMTADTSISTAYMELSRLRSDDTAVYYC (SEQ ID NO:77), WGQGTLVTVSS (SEQ ID NO:78), DIQMTQSPSTLSASVGDRVTITCKSS (SEQ ID NO:79), LAWYQQKPGKAPKLLIY (SEQ ID NO:80), TRESGVPSRFSGSGSGTEFTLTISSLQPDDFATYYC (SEQ ID NO:81), FGGGTKVEIK (SEQ ID NO:82), MAQVQLVQSGAEVKKPGASVKVSCKAS (SEQ ID NO:104), IHWVRQAPGQGLEWIGW (SEQ ID NO:105), EYAPKFQGRATMTADTSISTAYMELSRLRSDDTAVYYC (SEQ ID NO: 106), WGQGTLVTVSS (SEQ ID NO:107), MAQVQLVQSGAEVKKPGASVKVSCKAS (SEQ ID NO: 123), IHWVRQAPGQGLEWIGW (SEQ ID NO:124), EYAPKFQGRATMTADTSISTAYMELSRLRSDDTAVYYC (SEQ ID NO:125), WGQGTLVTV (SEQ ID NO:126),
or a polypeptide with at least 80%, 85%, 90%, 92%, 94%, 95%, 96%, 97%, 98%, or 99% amino acid sequence identity thereto.

5. The composition of claim 1 wherein the first, second, or third CDR, or any combination thereof, of variable region ii) is/are flanked by human Ig framework sequences.

6. The composition of claim 5 wherein the one or more human Ig framework sequences comprise DIQMTQSPSSLSASVGDRVTITCKSS (SEQ ID NO:55), LAWYQQKPGKAPKLLI (SEQ ID NO:56), TRESGVPSRFSGSGSGTDFTLTISSLQPEDFATYYC (SEQ ID NO:57), FGGGTKLEIK (SEQ ID NO: 58),, DIQMTQSPSSLSASVGDRVTITCRAS (SEQ ID NO:63), LAWYQQKPGKVPKLLIY (SEQ ID NO:64), TRESGVPSRFSYSGSGTDFTLTISSLQPEDVWTYYC (SEQ ID NO:65), FGQGTKLEIK (SEQ ID NO:66), DIVMTQSPDSLAVSLGERATINCKS (SEQ ID NO:71), LAWYQQKPGQPWKLLIY (SEQ ID NO:72), TRESGVPDRFSGSGSGTDFTLTISSLQAEDVYVYY (SEQ ID NO:73), FFQGTKVEIK (SEQ ID NO: 74), DIQMTQSPSTLSASVGDRVTITCKSS (SEQ ID NO:79), LAWYQQKPGKAPKLLIY (SEQ ID NO:80), TRESGVPSRFSGSGSGTEFTLTISSLQPDDFATYYC (SEQ ID NO:81), FGGGTKVEIK (SEQ ID NO: 82), DIQMTQSPSSLSASVGDRVTITCKSS (SEQ ID NO:100), LAWYQQKPGKAPKLLIY (SEQ ID NO:101), TRESGVPSRFSGSGSGTDFTLTISSLQPEDFATYYC (SEQ ID NO:102), FGGGTKLEIKENLYFQGAAALE (SEQ ID NO:103), DIQMTQSPSTLSASVGDRVTITCKSS (SEQ ID NO:108), LAWYQQKPGKAPKLLIY (SEQ ID NO: 109), TRESGVPSRFSGSGSGTEFTLTISSLQPDDFATYYC (SEQ ID NO:110), FGGGTKVEIKENLYFQGAAALE (SEQ ID NO:111), DIQMTQSPSSLSASVGDRVTITCKSS (SEQ ID NO:127); LAWYQQKPGKAPKLLIY (SEQ ID NO:128), TRESGVPSRFSGSGSGTDFTLTISSLQPEDFATYYC (SEQ ID NO:129), FGGGTKLEIKENLYFQGAAALE (SEQ ID NO:130), DIQMTQSPSTLSASVGDRVTITCKSS (SEQ ID NO:131), LAWYQQKPGKAPKLLIY (SEQ ID NO: 132), TRESGVPSRFSGSGSGTEFTLTISSLQPDDFATYYC (SEQ ID NO:133), FGGGTKVEIKENLYFQGAAALE (SEQ ID NO:134),
or a polypeptide with at least 80%, 85%, 90%, 92%, 94%, 95%, 96%, 97%, 98%, or 99% amino acid sequence identity thereto.

7. The composition of claim 1 further comprising a pharmaceutically acceptable carrier.

8. The composition of claim 1 wherein the variable region i) and the variable region ii) are joined together by a linker.

9. The composition of claim 1 which comprises the antigen binding fragment thereof.

10-19. (canceled)

20. A method to inhibit or treat depression or anxiety in a mammal, comprising: administering to a mammal a composition comprising an effective amount of a nucleotide sequence which encodes at least the variable region of a human heavy or light chain that binds human CCKBR, wherein the chain comprises:
i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO: 33); and/or
ii) a variable region comprising a first CDR comprising QSLLNSGNQKNY (SEQ ID NO: 34) operably linked to a second CDR comprising GAS operably linked to a third CDR comprising QNDHSYPYT (SEQ ID NO:36), or
an antibody or fragment comprising
i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO: 33); and/or
ii) a variable region comprising a first CDR comprising QSLLNSGNQKNY (SEQ ID NO: 34) operably linked to a second CDR comprising GAS operably linked to a third CDR comprising QNDHSYPYT (SEQ ID NO:36).

21. The method of claim 20 wherein the heavy chain is an IgG heavy chain.

22. The method of claim 20 wherein the light chain is an Igk light chain.

23. The method of claim 20 wherein the antibody fragment is administered.

24. The method of claim 23 wherein the fragment is Fab′ or scFv.

25. A method to inhibit or treat pain in a mammal, comprising: administering to a mammal a composition comprising an effective amount of a nucleotide sequence which encodes at least the variable region of a human heavy or light chain that binds human CCKBR, wherein the chain comprises:
i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO: 33); and/or
ii) a variable region comprising a first CDR comprising QSLLNSGNQKNY (SEQ ID NO: 34) operably linked to a second CDR comprising GAS operably linked to a third CDR comprising QNDHSYPYT (SEQ ID NO:36), or
an antibody or fragment comprising
i) a variable region comprising a first complementarity determining region (CDR) comprising GFNIKDYY (SEQ ID NO:31) operably linked to a second CDR comprising IDPENGDT (SEQ ID NO:32) operably linked to a third CDR comprising NAGGRFAY (SEQ ID NO: 33); and/or
ii) a variable region comprising a first CDR comprising QSLLNSGNQKNY (SEQ ID NO: 34) operably linked to a second CDR comprising GAS operably linked to a third CDR comprising QNDHSYPYT (SEQ ID NO:36).

26. The method of claim 25 wherein the mammal has neuropathic pain, was exposed to blunt force trauma or has traumatic brain injury.

27-29. (canceled)

30. The method of claim 20 wherein the composition is systemically administered.

31. The method of claim 20 wherein the composition is injected.

32. The method of claim 20 wherein the nucleotide sequence is in a viral vector.

33. The method of claim 20 wherein the composition inhibits dorsal root ganglion (DRG) neurons.
